(19) United States
(12) Patent Application Publication
(10) Pub. No.: US 2025/0000415 A1
(43) Pub. Date: Jan. 2, 2025

(54) METHOD AND SYTEM FOR PREDICTING NEURAL ACTIVATION AND PSYCHOPATHOLOGY IN SUBJECTS

(71) Applicant: University of Massachusetts, Boston, MA (US)

(72) Inventors: Adam Grabell, Amherst, MA (US); Lakshmi Manasa Kalanadhabhatta, Navi Mumbai (IN); Tauhidur Rahman, La Jolla, CA (US); Deepak Ganesan, Amherst, MA (US)

(73) Assignee: University of Massachusetts, Boston, MA (US)

(21) Appl. No.: 18/760,309

(22) Filed: Jul. 1, 2024

Related U.S. Application Data
(60) Provisional application No. 63/511,028, filed on Jun. 29, 2023.

(51) Int. Cl. A61B 5/16; G06F 3/01; G06V 40/16

(52) U.S. Cl. CPC A61B 5/165; G06F 3/013; G06V 40/171; G06V 40/176

(57) ABSTRACT

A method of predicting neural activation and psychopathology, indicative of ADHD and mood and anxiety disorders in a subject, such as children, is disclosed. The method may be implemented via computer software on a tablet computer, laptop or desktop computer, an input device, such as touchscreen, keyboard or mouse, and a camera. The camera is positioned to capture the subject's face in the frame of the camera. A frustration-inducing activity, such as a game, to the subject, where the subject received positive or negative feedback based on the selections they make in a game. The subject's reactions to the feedback are captured and parsed by the software, to determine the subject's emotional reaction to feedback. Reactions such as eye gaze, head pose, and facial expression may be used. Over a number of trials of the game, a prediction may be made for the neural activation and psychopathology of the subject.

### CROSS-REFERENCE TO RELATED APPLICATIONS

This patent document claims priority to earlier filed U.S. Provisional Patent Application Ser. No. 63/511,028, filed on Jun. 29, 2023, the entire contents of which are incorporated herein by reference.

### STATEMENT REGARDING FEDERALLY SPONSORED RESEARCH OR DEVELOPMENT

This invention was made with government support under Grant #NIMHIK23MH111708 awarded by the National Institutes for Health and Grant #s 839999, 1951928, 1815347 awarded by the National Science Foundation. The government has certain rights in the invention.

### BACKGROUND OF THE INVENTION

### 1. Field of the Invention

The present patent document is directed generally to methods for early detection of psychopathology, such as ADHD, Oppositional Defiant Disorder, mood disorders, anxiety disorders, and severe irritability, by way of example, and associated neural activity, in subjects, such as preschool age children, and more particular an improved method and system for the detection thereof.

### 2. Background of the Related Art

Emotion dysregulation in early childhood is known to be associated with a higher risk of several psychopathological conditions, such as ADHD, conduct, mood and anxiety disorders. In developmental neuroscience research, emotion dysregulation is characterized by low neural activation in the prefrontal cortex during frustration.

Emotion regulation—the ability to modulate the duration, valence, or intensity of an emotional experience [61]—is one of the most widely studied topics in neuroscience and psychology. Researchers have examined emotion regulation capabilities among infants [26], young children [34, 56], adolescents [25], as well as adults [17]. In particular, poor emotion regulation, particularly in response to negative emotional challenges such as frustration, is a core feature of some of the most commonly diagnosed psychological disorders that persist across the lifespan, such as attention deficit hyperactivity disorder (ADHD) [13, 114], childhood depression [80], pediatric bipolar disorder [81], and a range of other psychopathological disorders [65, 91, 92, 99].

Problems with emotion regulation emerge early in life, are closely tied to early-onset psychological disorders that disrupt children's functioning, persist into later stages of development, and exert an enormous financial burden on society [16]. There has been particular interest in studying emotion regulation during the preschool years, when this ability develops rapidly and has a profound effect on children's capacity to function adaptively in school, home, and social environments. Early childhood emotion regulation is essential for academic achievement [60] as well as for the formation of early friendships [46]. Poor emotion regulation predicts over a dozen DSM-5 disorders and is the most common reason young children are referred to psychological services [8].

Despite the urgent need to identify these psychological disorders early, most commonly used diagnostic instruments such as standardized questionnaires and semi-structured clinical interviews are long or expensive to administer [88] and have surprisingly poor accuracy compared to diagnostic tools used in physical medicine [106, 115], with area under curve (AUC) values in the 0.7-0.8 range [21, 69]. The difficulty in achieving high diagnostic accuracy stems in part from the fact that signs of problematic emotion regulation, such as temper tantrums, are difficult to distinguish from normative misbehavior young children commonly exhibit [123]. This creates a ‘when to worry’ problem where caregivers lack guidelines to determine the severity and clinical significance of a child's behavior. Furthermore, attaining a psychological diagnosis typically requires families to overcome several barriers in order to seek clinical care, including awareness, cost, and labor burdens [24]. “Gold standard” diagnostic tools also require specialized training and clinical services, are extremely time intensive, and therefore difficult for clinicians to implement in community settings [74, 105].

There is an opportunity for next-generation diagnostic instruments to identify not just psychological disorders, but abnormalities in the neurobiological mechanisms that drive them. Neuroimaging work over the past few decades has led to major advances in identifying the neural mechanisms underpinning the emotion regulation response and driving symptoms of psychopathology [59]. Several studies link decreased neural activation in the lateral Pre-Frontal Cortex (LPFC) to poorer emotion regulation and higher aggressive behavior [32], and dysfunctional LPFC activation to depression and ADHD [36, 78]. However, neuroimaging via functional Magnetic Resonance Imaging (fMRI) is expensive and also especially unsuitable for young children, as it requires them to lie completely still in the scanner for extended periods of time. Techniques such as functional Near-Infrared Spectroscopy (fNIRS) provide a more comfortable alternative but remain prohibitively expensive for diagnostic screening at a large scale. Therefore, most neuroimaging studies are restricted to in-laboratory observations of relatively few participants and mental health practitioners have to rely on questionnaires for diagnostic purposes.

Accordingly, there is a need in the art for improved methods for detecting psychopathology, such as ADHD and anxiety disorders by way of example, in subjects, such as preschool age children.

### SUMMARY OF THE INVENTION

This patent document discloses a novel multi-scale instance fusion framework to develop a set of classifiers trained on behavioral markers during emotion regulation. The system and method disclosed herein successfully predicts activation levels in the prefrontal cortex with an area under the receiver operating characteristic (ROC) curve of 0.85, which is on par with widely-used clinical assessment tools. Further, clinical and non-clinical subjects are classified based on their psychopathological risk with an area under the ROC curve of 0.80. The method and system's predictions are consistent with standardized psychometric assessment scales, supporting its applicability as a screening procedure for emotion regulation-related psychopathological disorders.

In this work, we report on an exploratory study with 94 participants aged 3.5 to 5 years, investigating whether behavioral measures automatically extracted from facial videos can predict frustration-related neural activation and differentiate between low- and high-risk individuals. While this work is targeted at pre-school age children, because early detection of conditions can lead to early treatment, it should be understood that the methods herein may be extended to other populations and conditions.

We investigate whether it is possible to leverage automatically extracted behavioral features from video cameras to develop novel and more informative tools to support clinical diagnosis. Prior research suggests that neural activation during emotion regulation could be indirectly measured via cameras in two ways. The first is via facial expressions, which researchers have found to be strong behavioral correlates of emotion regulation in children [33, 108, 137]. Certain expressions such as a Duchenne smile (see [47]) or a frown during emotion regulation have been shown to be correlated with neural activity in the lateral and medial prefrontal cortex and the amygdala [58, 64, 103]—regions of the human brain known to be associated with emotion regulation [22, 37, 114, 119]. The second is via eye and bodily movement-related measures, which have been identified as potential biomarkers for ADHD diagnosis [7, 82, 83]. Gaze fixations have also been shown to predict neural activation during emotion regulation [120].

### BRIEF DESCRIPTION OF THE DRAWINGS

These and other features, aspects, and advantages of the present invention will become better understood with reference to the following description, appended claims, and accompanying drawings where:

FIG. 1 is a flowchart of an exemplary embodiment of the method and system described herein;

FIG. 2A is an exemplary screenshot of an embodiment of a first step of the method and system described herein, where the subject is introduced to the concept of the game;

FIG. 2B is an exemplary screenshot of an embodiment of a second step of the method and system described herein, where the subject selects the makes a choice;

FIG. 2C is an exemplary screenshot of an embodiment of a third step of the method and system described herein, where the subject is shown positive of negative feedback based on the choice made in the second step;

FIG. 3 is an illustration of an exemplary embodiment of the system described herein;

FIG. 4 is an illustration of Table 1 showing a comparison of an embodiment of EarlyScreen with previous works relating behavioral measurements to neural activation or psychopathology;

FIG. 5 is an illustration of an outline of the emotion induction task completed by the participantsm, where the upper panel shows the design of the Incredible Cake Kids game, which is played 30 times (six blocks of 5 trials each), and the lower panel shows the order of positive and negative trials;

FIG. 6 is an illustration of an experimental setup showing a child participant in the laboratory, where the child participant is positioned in front of a touchscreen computer screen and is wearing the fNIRS probe for neuroimaging;

FIG. 7A is a front view of an illustration showing placement of the NIRS optodes on the prefrontal cortex, where the red and purple dots show source and detector optode positions respectively, and the lines between them show measurement channels;

FIG. 7B is a left side view of an illustration showing placement of the NIRS optodes on the prefrontal cortex, where the red and purple dots show source and detector optode positions respectively, and the lines between them show measurement channels;

FIG. 8A is an illustration of a child participant with a neutral face;

FIG. 8B is an illustration of a child participant with a simple facial masking expression, where the blue arrow shows contraction of the lip corner puller (AU 12) producing a smile;

FIG. 8C is an illustration of a child participant with a complex facial masking expression during negative feedback, where the blue arrow shows contraction of the lip corner puller (AU 12) producing a smile, and the red arrow shows contraction of the upper lip raiser (AU 10) producing a sneer, and deepening the nasolabial fold;

FIG. 9 is an illustration of an exemplary embodiment of a Multi-scale Instance Fusion (MIF) framework, where the MIF framework is an ensemble of two parallel classification pipelines operating on single and multiple instance features respectively, and where the predicted probabilities from both pipelines are fused to output the final class label for the participant;

FIG. 10 is an illustration of Table 2, showing classification performance for low vs. normal activation detection using baseline Single Instance Learning (SIL) pipelines and the Multi-scale Instance Fusion (MIF) model;

FIG. 11 is a graph of the receiver operating characteristic (ROC) curve of the best performing model for predicting PFC activation. The area under the ROC curve is 0.85;

FIG. 12 is a normalized confusion matrix showing classification results of the best performing model for predicting PFC activation;

FIG. 13 is an illustration of Table 3, showing classification performance for detecting clinical vs. non-clinical participants using baseline Single Instance Learning (SIL) pipelines and the proposed Multi-scale Instance Fusion (MIF) model;

FIG. 14 is a graph of the receiver operating characteristic (ROC) curve of the best performing model for predicting clinical dis-order status. The area under the ROC curve is 0.80;

FIG. 15 is a normalized confusion matrix showing classification results of the best performing model for predicting clinical disorder status;

FIG. 16A is a graph of a distribution of scores for CBCL Externalizing, after correcting for false discovery rate using the Benjamini-Hochberg procedure at significance level α=0.05;

FIG. 16B is a graph of a distribution of scores for ADHD Inattention, after correcting for false discovery rate using the Benjamini-Hochberg procedure at significance level α=0.05;

FIG. 16C is a graph of a distribution of scores for ADHD Hyperactivity, after correcting for false discovery rate using the Benjamini-Hochberg procedure at significance level α=0.05;

FIG. 16D is a graph of a distribution of scores for MAP-DB Temper Loss, after correcting for false discovery rate using the Benjamini-Hochberg procedure at significance level α=0.05;

FIG. 17 is an illustration of Table 4, showing correlation between predicted probability of being classified as ‘Clinical’ and scores on different psychopathological symptom scales, where higher scores indicate abnormality on all scales, and where the table shows p values that are not corrected for multiple comparisons, and * indicates significance after correcting for the false discovery rate using the Benjamini-Hochberg procedure at significance level α=0.05;

FIG. 18 is a chart showing average area under ROC curve over 5 folds for different values of λ in the best performing MIF model for predicting neural activation levels;

FIG. 19 is a chart showing average area under ROC curve over 5 folds for different values of λ in the best performing MIF model for predicting clinical disorder status;

FIG. 20A is a graph showing performance of the neural activation classification model by gender;

FIG. 20B is a graph showing performance of the neural activation classification model by age;

FIG. 20C is a graph showing performance of the neural activation classification model by race;

FIG. 21A is a graph showing performance of the psychopathology classification model by gender;

FIG. 21B is a graph showing performance of the psychopathology classification model by age;

FIG. 21C is a graph showing performance of the psychopathology classification model by race;

FIG. 22 is an illustration of survey respondents' attitudes towards current diagnostic practices and beliefs about the potential utility of other sources of data;

FIG. 23 is an illustration of survey respondents' feedback on the utility of EarlyScreen and potential concerns for deployment;

FIG. 24 shows an exemplary embodiment of a frustration-inducing task on a Windows Surface Pro tablet according to the present invention;

FIG. 25 is an illustration of Table 5, showing profiling characteristics of the frustration-inducing task on a Windows Surface Pro tablet.

### DESCRIPTION OF THE PREFERRED EMBODIMENT

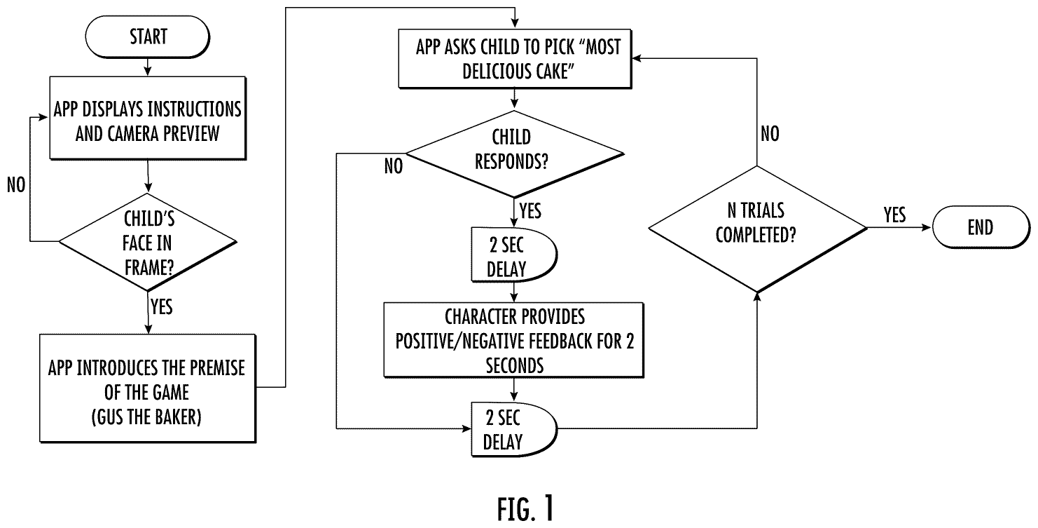

Referring to FIG. 1, and as will be described in greater detail below, the present patent document discloses a method of predicting neural activation and psychopathology, indicative of ADHD and mood and anxiety disorders in subjects, such as children. In one embodiment, the method may be implemented via computer software on a tablet computer, with a processor, memory, storage, a touchscreen display, and rearward facing camera. However, a laptop or desktop computer may be used with external input devices, such as keyboard and mouse, for example, or touchpad, may be used. Additionally, the camera may be integrally or externally mounted. Regardless of the mounting of the camera, the subject's face must be present in the frame of the camera so that the subject's expression may be captured.

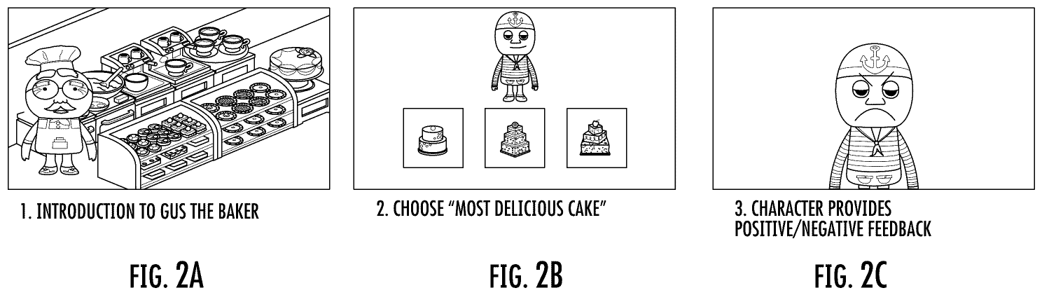

Referring to FIGS. 1 and 2A, the software provides a frustration-inducing activity, such as a game, to the subject. In one embodiment, the premise of the game may be “Gus the Baker,” which provides a make-believe bakery having a variety of “delicious cakes” or other deserts and foodstuffs. However, it should be understood that the “game” may be made more engaging for other populations, such as pre-teens, teens, adults, and the elderly.

Referring to FIGS. 1 and 2B, the subject is asked to pick the “most delicious cake.” The software waits for the subject to respond.

Referring to FIGS. 1 and 2C, once the subject, with the input device, selects the cake that they believe is the most delicious, the software waits a moment, such as two seconds, and then presents feedback to the subject, which can comprise positive or negative feedback via a character, animations, and/or music and sound effects.

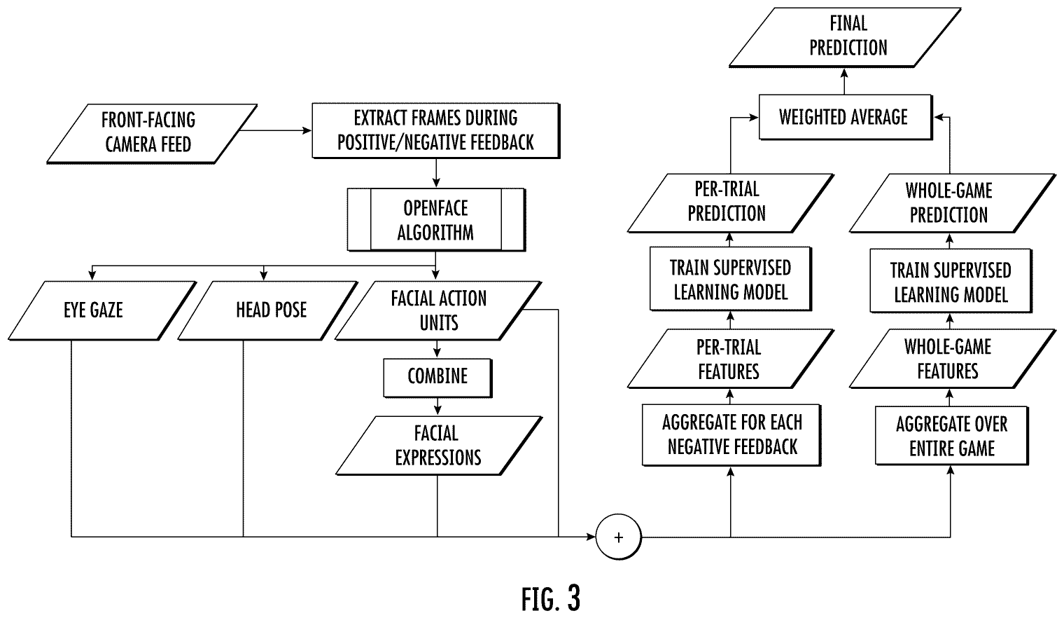

Referring to FIG. 3, the camera captures the subject' reaction to the feedback. The software, running on the computer, causes the processor to extract frames from the subject's reaction and attempts to determine characteristics of the subject's reaction and facial expression in correlation to the feedback. In one embodiment, the subject's eye gaze, head pose and facial expressions are determined. By comparing the subject's reaction over a number of trails of the game, in combination with the type of feedback received, a weighted average and final prediction neural activation and psychopathology can be made for the subject.

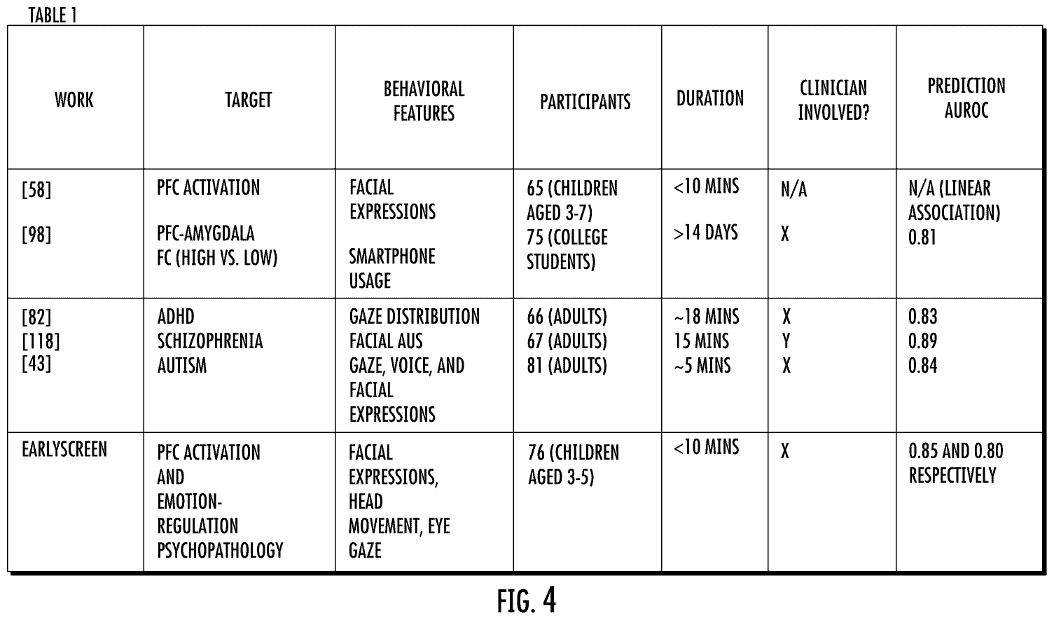

Referring to FIG. 4, Table 1 summarizes the novelty of an embodiment of the system and method described herein, called EarlyScreen, compared to previous work: we focus on predicting both neural activation and psychopathology in a preschool-aged population and achieve competitive prediction performance in this challenging context. However, it should be understood that the methods herein may be extended to other populations and conditions.

### Experimental Results

### Emotion Induction Task

Ninety-four participants, ages 3.5 to 5 years old (Mean age=4.05, SD=0.73%), participated in the present study (54.3% male, 45.7% female; 75% White, 9.8% Black or African American, 9.8% multiracial, 2.2% Asian, and 3.3% “choose not to respond”). Families were recruited through social media platforms and community outreach and completed a 5- to 10-minute phone screening to determine eligibility for participation. Exclusionary criteria included psychotic symptoms, an existing diagnosis of developmental or intellectual disabilities, a history of head trauma with loss of consciousness, and the inability to speak or understand English. One parent of the participant completed informed consent. The present procedure was part of a larger study assessing behavioral, neural, and physiological predictors of irritability and emotion regulation in young children. Families received a $60 compensation for participating and children received a certificate and a small toy. This research study was approved by the Institutional Review Board of the University of Massachusetts Amherst.

### Emotion Induction Task

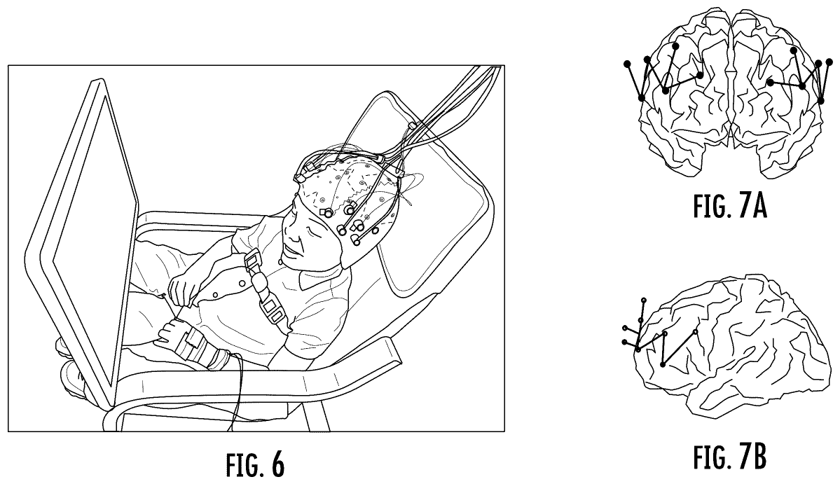

Participants completed a computerized frustration-inducing task designed for preschool children titled “Incredible Cake Kids” [57] on a touch screen monitor while facial video and prefrontal cortex activation were recorded simultaneously (see FIG. 6). The task premise was that a virtual bakery needs the child's help baking virtual cakes for different customers. Children were instructed to choose the “most delicious cake” for each customer. They were also told that some children are better than others at choosing the most delicious cake and that they would be evaluated on their performance. Before starting the task, the children watched an instructional video and practiced playing the game.

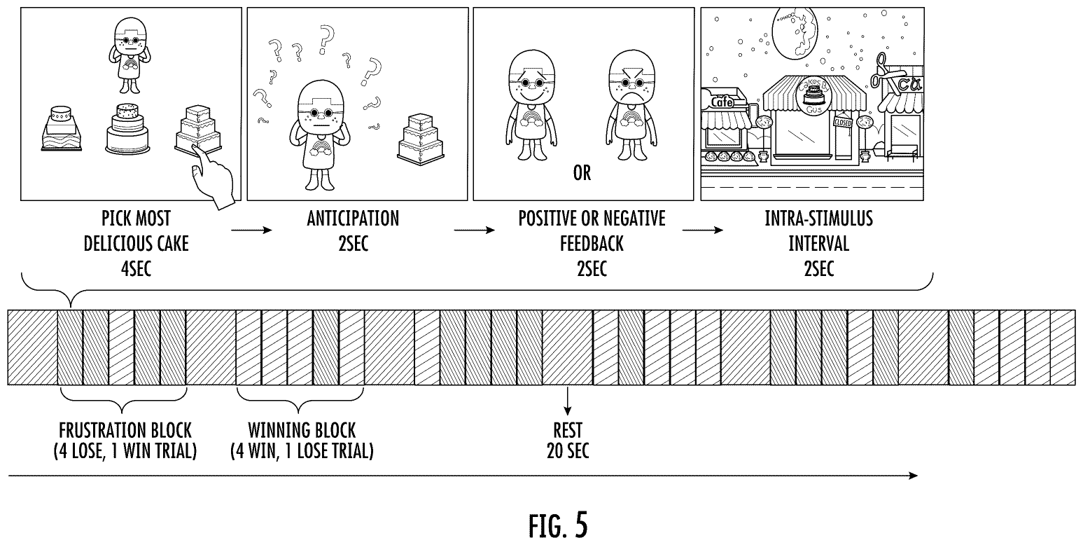

The children then completed 30 trials of the task. For each trial, a virtual customer appeared on the screen along with three virtual cakes for four seconds in which children picked the “most delicious” cake, followed by two seconds of anticipation, and two seconds of positive (e.g., happy) or negative (e.g., grumpy) feedback (see FIG. 5). Unbeknownst to the child, virtual customers provided predetermined positive or negative feedback, which were organized into three negative (four negative and one positive trials grouped together) and three positive blocks (four positive and one negative trial), separated by 20-second rest periods between blocks. The task lasted approximately 10 minutes, and the entire session was video recorded for further analysis. On average, participants selected a cake in 25.77 (SD=4.50) of the 30 trials, showing that the children were sufficiently engaged in the game.

Neural Activity via Functional Near-Infrared Spectroscopy (fNIRS)

Participants' neural activity was measured during the emotion induction task through non-invasive optical imaging via functional Near-Infrared Spectroscopy (fNIRS). In recent years, fNIRS has emerged as an alternative to traditional neuroimaging techniques such as EEG (which offers poor localization of brain activity) and fMRI. Compared to fMRI, fNIRS is better suited to measure neural activity in infants and children, as it is more robust to motion artifacts. Moreover, it allows the subject's face to be observed more clearly compared to fMRI, making it easier to record simultaneous brain and facial activity.

We used a NIRx NIRScout imaging system, with an fNIRS probe consisting of eight light-source emitters with 760 nm and 850 nm LED lights and four detectors. The sources and detectors were attached to an elastic cap with an average inter-optode distance of 3 cm. The international 10-20 coordinates [73] were followed to place the probe, aligning the interior medial corner of the probe with the prefrontal midline sagittal plane FpZ and extending it over Brodmann areas 10 (ventrolateral prefrontal cortex) and 46 (dorsolateral prefrontal cortex) on both the left and right hemisphere. The probe constituted 10 channels that were grouped into a single region of interest (ROI)—the lateral prefrontal cortex (LPFC)—similar to previous work (e.g., [58]). The ten measurement channels resulting from this placement are shown in FIGS. 7A and 7B.

The fNIRS data was analyzed using the NIRS toolbox 1. Data were recorded at 7.81 Hz and downsampled to 4 Hz for further analysis. The raw intensity per channel was used to calculate the delta optical density (ΔOD), or the change in light absorption through brain tissue over time. This is defined as

$$Δ OD = - log ( I / I 0 )$$

where I is the recorded signal intensity and I0 is the reference baseline intensity. We then used ΔOD to calculate changes in oxyhemoglobin and deoxyhemoglobin (ΔHbO2 and ΔHbR, respectively) using the modified Beer-Lambert law [71]. The change in oxyhemoglobin (ΔHbO2) is modeled as a generalized linear model (GLM) for each experimental condition (Positive, Negative, and Rest blocks) per subject, where

$$Δ HbO 2 = X * β + ϵ$$

The design matrix X is given by a convolution of the stimulus timings with the canonical hemodynamic response function, which defines the shape of the expected change in ΔHbO2 following a stimulus.

The GLM is fit using an autoregressive iteratively reweighted least squares approach, which corrects for motion artifacts and serially correlated errors due to underlying physiology [12]. We thus estimated the coefficients, β, for each channel during each condition. Finally, a single beta value is calculated by averaging the coefficients across all channels within the ROI per condition for each participant. The baseline beta for the Rest condition is subtracted from the values for the Positive and Negative conditions, giving us a measure of the magnitude of the relative evoked hemodynamic response for each condition. This serves as the ground truth measure of the level of neural activation during that block—a higher beta value indicates higher neural activity. In this work, we focus primarily on neural activity during frustration, that is, during negative blocks. Higher beta values during negative blocks indicate better emotion regulation, and low beta values signify poorer emotion regulation and greater risk of psychopathology [59].

### Psychopathology Measures

In order to test our hypothesis of detecting diagnostic status from behavioral data, we also obtained participants' scores on a variety of clinically validated psychopathology measures.

First, caregivers reported their children's frequency of ADHD symptoms via the ADHD Rating Scale-5 Home Version [44]. The rating form consisted of a 4-point Likert scale (0=Never or Rarely, 1=Sometimes, 2=Often, and 3=Very Often), by which parents indicated the frequency of each behavior. Caregivers were instructed to select the number that best represented their child's behavior over the past six months. Following data collection, the 18 behavior items were subdivided into two subscales, ADHD Inattention (e.g., “Has difficulty sustaining attention”) and ADHD Hyperactivity (e.g., “Has difficulty waiting his or her turn”). Children who scored 1-5 on these subscales were considered to be outside the clinical range for ADHD, and children who scored 6 or above were considered to be in the clinical range.

The Temper Loss subscale from the Multidimensional Assessment Profile for Disruptive Behavior (MAP-DB; [121]) was also used as a measure of child irritability. The MAP-DB aims to differentiate irritability in the normative range from irritability in the clinical range. The Temper Loss subscale specifically measures irritable mood (e.g., “Become frustrated easily”) and tantrums (e.g., “Lose temper or have a tantrum during daily routines”) as factors of irritability. Caregivers reported their child's irritability frequency over the past month via a 6-point Likert scale (1=Never, 2=Rarely (less than once per week), 3=Some (1-3 days of the week), 4=Most (4-6 days of the week), 5=Every day of the week, and 6=Many times each day). Children who received a total score of 42.5 or greater were considered to be in the clinical range.

Caregivers also reported their child's behaviors using the Child Behavior Checklist for ages 1.5 to 5 (CBCL; [3]). The CBCL comprised 99 items that were rated via a 3-point Likert scale (0=Not True, 1=Somewhat or Sometimes True, and 2=Very True or Often True). Caregivers were instructed to select the number that best describes their child's behavior in the present or within the past two months. Items were subdivided into two subscales, Internalizing (i.e., symptoms related to anxiety and mood disorders) and Externalizing (i.e., symptoms related to disruptive behavior disorders and ADHD) behaviors. Children who scored a 65 or above in one or both of these subscales were considered to be in the clinical range.

Finally, we aggregated the scores of the participants on all scales to categorize them as within or outside the clinical range. Children were considered to be in the clinical range for psychopathological risk if they scored above the cut-off thresholds on at least one of the ADHD Inattention, ADHD Hyperactivity, CBCL Externalizing, or MAP-DB symptom scales. We do not consider the CBCL Internalizing Behavior subscale due to its poorer discriminatory power in children of our target age (internalizing disorders are also not prevalent at this age) [40]. Children scoring below the clinical threshold on all scales were considered to be in the non-clinical range overall. We later attempted to differentiate between clinical vs. non-clinical participants using behavioral features from videos.

Behavioral Features Extracted from Videos

We used two high definition cameras—one trained on the child's face and the second one at an angle—to record children's facial expressions and head/upper body movement during the emotion regulation task. Based on observations from prior work, we extracted facial expressions, eye gaze, and head movement measures from the collected videos.

To extract visible facial expressions, we recorded facial movements using the Facial Actions Coding System (FACS) [54]. FACS allows visible facial expressions to be coded based on anatomical facial muscle movements categorized as Action Units (AUs). We used the OpenFace 2.0 library [10] for AU detection, which contains models to predict the presence or intensity of various AUs in images and videos. In this study, we extracted 18 facial AUs (see Appendix A) from the video stream obtained from the camera directly pointed toward the child's face. We disregard the camera placed at an angle since it fails to capture the entire face of the subject and sometimes also includes the experimenter in the frame. Since our study focuses on emotion regulation during frustration, we focused on facial expressions during and immediately following positive or negative feedback received by the children in each trial. We extracted 4-second-long video segments immediately after the feedback event, resulting in 30 segments (15 positive and 15 negative feedback) per participant.

From the same feedback segment videos, we also extracted the eye gaze direction and head pose per frame using OpenFace. The library computes a 3-dimensional gaze vector for each eye as well as an averaged gaze angle vector for both eyes in two dimensions. We used the latter measure to compute the change in gaze angle per unit time. To estimate head movement, we used OpenFace to calculate the position of the child's head within the frame relative to the (stationary) camera in 3D. We estimated the change in head position per unit time by computing the displacement between consecutive frames.

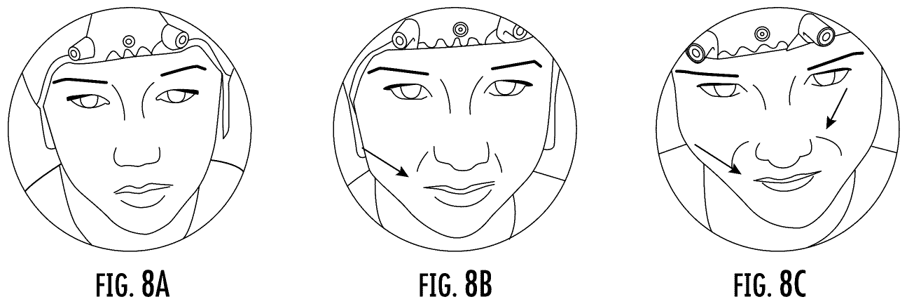

We also used the facial AUs to detect categories of facial expressions that have been shown to be relevant to emotion regulation [58]. The first category comprises negative expressions, characterized by the presence of one or more negative action units without the presence of any positive action unit. The second category consists of positive expressions which are considered to be exhibited when the child is masking their frustration—this is characterized by the presence of AU 12 (lip corner puller). FIGS. 8A-8C shows a child demonstrating masking expressions of two kinds—simple masking, where AU 12 is not accompanied by any co-occurring negative AUs, and complex masking, where AU 12 is accompanied by the negative AU 10 (upper lip raiser). Both negative and positive (masking) expressions may be accompanied by eye constriction (AU 6). We grouped the relevant AUs extracted from OpenFace into positive/negative expressions with/without eye constriction in order to analyze their association with neural activation. See Appendix A for a list of all AUs extracted for the study, along with their categorization into positive and negative expressions.

### Multi-Scale Instance Fusion Framework

As described above, EarlyScreen aims to utilize behavioral features extracted from videos to predict (i) neural activation levels in the PFC, as well as (ii) psychopathological disorder status among preschool children. The key challenge in doing so is the limited number of labels (one per individual) in our dataset, providing only coarse-grained subject-level information. At the same time, the frustration-inducing task completed by our participants comprises of multiple trials (as shown in FIG. 5). This leads to the availability of up to 30 observations of each participant's behavioral response (15 positive and 15 negative feedback trials), which were recorded in the form of facial videos. Behavioral responses from each of these trials can be thought of as independent episodes of data containing fine-grained information.

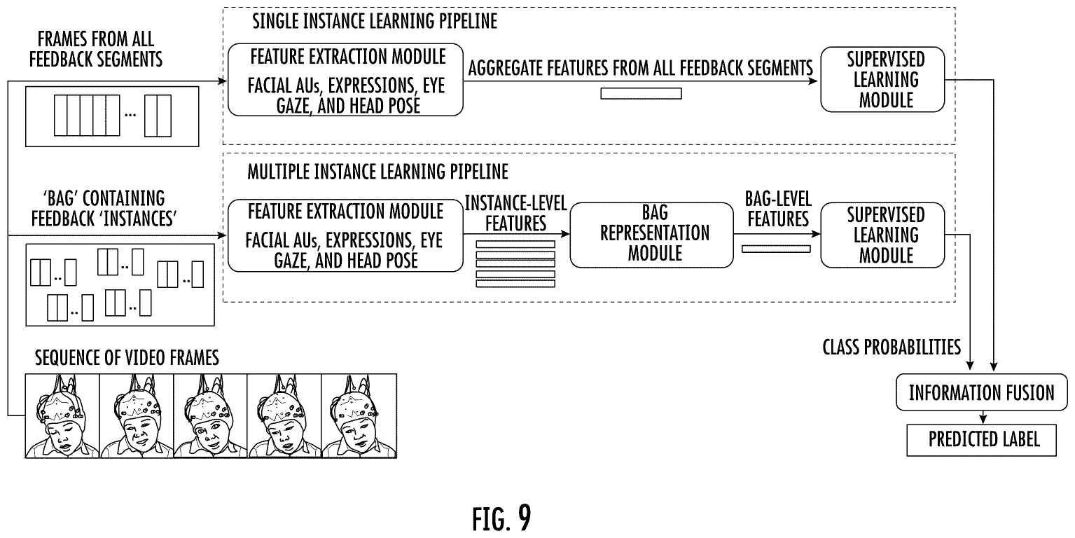

In this paper, we propose a Multi-scale Instance Fusion (MIF) framework to bridge the gap between the availability of multiple independent behavioral observations and individual-level neural activation or psychopathology labels. The MIF framework harnesses both fine-grained information from each trial as well as coarse-grained information from the overall session to characterize an individual. It combines (i) a Multiple Instance Learning (MIL) Pipeline learning subject-level features from each instance of feedback with (ii) a corresponding Single Instance Learning (SIL) Pipeline operating on coarse-grained features extracted by aggregating all feedback segments. FIG. 9 shows our proposed MIF architecture, which is an ensemble model consisting of these two components fused together in order to obtain the final predicted label. We now describe each component in the MIF framework in more detail.

### Multiple Instance Learning Pipeline

Multiple Instance Learning (MIL) [42] is a weakly supervised machine learning framework where each sample in the dataset is a bag Bi with a single label Yᵢ. The bag contains nᵢ instances represented as xᵢⱼ, j=1, 2, . . . , nᵢ, and the labels yᵢⱼ for individual instances are generally unavailable.

The MIL framework has been applied to a number of learning problems in prior literature, each with its own set of assumptions. The standard MIL assumption is defined as a classification problem in which negative and positive instances are grouped into bags such that negative bags contain only negative instances, whereas positive bags contain at least one positive instance [42]. Formally, the bag label Yᵢ can then be represented in terms of the instance labels yᵢⱼ as

$${Y}_{i} = \begin{matrix}{t}^{} + 1 & if ∃ {y}_{ij} : {y}_{ij} = + 1 \\ - 1 & if ∀ {y}_{ij} : {y}_{ij} = - 1\end{matrix}$$

In the above scenario, to classify a bag as positive, it is sufficient to identify one positive instance within the bag. On the other hand, the collective MIL assumption refers to problems where the bag label Yᵢ is defined by more than one instance label, or by none of the instance labels. Carbonneau et al. [28] provide a detailed review of MIL frameworks under different assumptions. Several approaches have been proposed to learn both bag- and instance-level labels under the MIL formulation. These include approaches that model instance labels as hidden variables [139], or extensions of regular supervised learning algorithms to an MIL space (e.g., [6, 138]).

The MIL framework can also be adapted and applied to problems that do not satisfy the standard assumption described above (e.g., [29, 130]). Some methods proposed for this setting include bag distance-based techniques [125], bag kernel representations [55], or bag dissimilarity mappings [116]. Another approach is to propagate instance-level features to a bag-level feature space and then utilize standard supervised learning techniques for the final classification [85].

The classification problems in our work can be translated to a Multiple Instance Learning (MIL) problem with relaxed assumptions where each individual is represented as a “bag” with a single label for neural activation levels or psychopathological disorder status. As described earlier, our task contains 30 trials—15 positive and 15 negative feedback—grouped into six blocks. Each of these trials can be thought of as an “instance” within the bag. A feature extraction module extracts instance-level features, which in our case include facial AUs, expressions, eye gaze, and head pose. These instance-level features are collectively used to compute a bag representation, and a supervised learning module uses this representation to predict class probabilities. This makes up what we define as a Multiple Instance Learning (MIL) pipeline.

### Single Instance Learning Pipeline

While the MIL setup described above allows us to utilize each observed positive/negative feedback instance on its own, it fails to capture overall behavioral characteristics from the participant's multiple trials. This is in turn achieved by the Single Instance Learning (SIL) Pipeline, which uses coarse-grained features from facial video frames belonging to all feedback segments in the frustration inducing task. These features are extracted by a feature extraction module and act as inputs to a supervised learning module that outputs predicted probabilities for each class. The predicted probabilities from both SIL and MIL pipelines are fused together by weighted averaging, resulting in a final probability assigned to each class that is used to determine the predicted label.

### Feature Extraction Module

The feature extraction module is part of both the SIL and MIL pipelines in the MIF framework. Features computed over all feedback segments are passed to the supervised learning module in the SIL pipeline, whereas features are computed for each instance separately and passed to the bag representation module in the MIL setting. More specifically, the feature extraction module within the SIL pipeline generates a single representation for all data segments associated with a negative or positive feedback segment. In the MIL pipeline, features are extracted separately from each distinct 4-second feedback segment.

Given a set of video frames, the feature extraction module first utilizes the OpenFace library to compute frame-level presence of facial AUs, head pose, and gaze direction. We then identify the presence of each predefined facial expression (positive/negative expressions with eye constriction, positive/negative expressions without eye constriction, and all positive/negative expressions) in each frame. We consider six different feature sets that are extracted by the Feature Extraction Module:
AU: Includes the presence (present/absent), duration (fraction of time present), and average intensity of each AU extracted from OpenFace (see Table 6) within a given segment.
Movement: Includes mean shift in eye gaze per unit time, standard deviation of the shift in eye gaze per unit time, mean shift in head pose per unit time, and standard deviation of the shift in head pose per unit time within a given segment.
Facial Expressions: Includes duration of positive expressions (with eye constriction, without eye constriction, total) and negative expressions (with eye constriction, without eye constriction, total) within a segment.
AU+Movement: Combination of the AU and Movement feature sets.
AU+Facial Expressions: Combination of the AU and Facial Expressions feature sets.
AU+Movement+Facial Expressions: Combination of the AU, Movement, and Facial Expressions feature sets.

### Bag Representation Module

The bag representation module within the MIL pipeline is responsible for learning a mapping from a bag's representation Bi in the instance-level feature space to a bag-level representation Bϕ. The choice of mapping depends on a number of key considerations. In the context of our work, these includei the following:
a. The label space for the bags and the label space for the instances are different. In our application scenario, the bag labels are assigned based on the individuals' observed neural activation over the entire task or based on the psychopathology symptom scores from questionnaires. On the other hand, instances within a bag correspond to different trials of the same participant and therefore do not have a neural activation or psychopathology label of their own. Given this distinction, our MIL pipeline should be applicable irrespective of whether a relationship exists between the bag and instance label spaces, making our framework more general than the standard MIL formulation.
b. For simplicity, we assume that the instances in a bag are not temporally ordered, i.e., each trial is independent of the others and the order of trials is not relevant. Therefore, our instance-to-bag mapping should be permutation invariant.
c. The number of observed trials for each participant can be different. Missing trials can occur if a child does not select a cake and therefore does not receive feedback in a trial, or if their face is occluded from the camera and we are unable to capture data. Therefore, there should be no constraints on the number of instances in each bag.

We evaluated a number of bag representation functions from MIL literature based on these considerations, and selected a subset of choices satisfying the above criteria. These mapping functions are listed below (see Appendix B for mathematical definitions):
Mean Mapping: aggregation of features by averaging across instances.
Minimax Mapping: representation using minimum and maximum values of each feature across instances. Polynomial Minimax Kernel [55]: representation of bag similarities as a polynomial kernel based on Minimax mapping.
MInD Mapping [30]: representation using a vector of bag-to-bag dissimilarities.
CCE Mapping [140]: bag representation based on instance-level clustering.
MILES Mapping [29]: representation using a vector of bag-to-instance similarities.
Discriminative Bag Mapping (aMILGDM) [133]: representation using a vector of similarities between each bag and each instance in a discriminative instance pool.

The above mapping algorithms are a non-exhaustive list of choices that can be utilized as part of the bag representation module in the MIF framework. They can be substituted by other algorithms that result in the transformation of a bag Bi containing xij⊆Rd, j=1, 2, . . . , nᵢ into a representation Bᵢϕ in the bag feature space. The resulting bag representation Bᵢϕ is used as input to a supervised learning module as shown in FIG. 5. We later discuss the effectiveness of more complex bag representations over baselines such as Mean and Minimax mapping below.

### Supervised Learning Module

Both the SIL and MIL pipelines in the MIF framework contain a supervised learning module, the primary component of which is a standard supervised machine learning classifier. We limit the choice of classifiers to probabilistic classifiers that predict a conditional distribution (Pr Y=y|X) for each class y. In addition to a classifier, the module may contain submodules for preprocessing input features (either coarse-grained features in the SIL pipeline or the bag representations in the MIL pipeline), including standardization or normalization, feature selection, under- and over-sampling to deal with class imbalance, etc.

### Information Fusion and Prediction

The final component of the MIF framework is the information fusion module that takes as inputs the predicted class probabilities from the SIL and MIL pipelines and aggregates them by weighted averaging to output a final probability. Specifically, the final conditional probabilities are calculated as

$${Pr ( Y = y \vert X ) = λ * {\{ Pr ( Y = y \vert X ) \}}_{SIL} + ( 1 - λ ) * [ Pr ( Y = y \vert X ) \}}_{MIL}$$

The predicted label is then calculated based on this aggregate probability:

$${y}^{⋀} = arg max Pr ( Y = y \vert X )$$

The weighted fusion proposed above ensures that our MIF framework performs at least as well as the SIL pipeline alone (when λ=1) or the MIL pipeline alone (when λ=0). We hypothesize that the fusion of the two would improve classification performance by learning both inter-instance and inter-bag variability. As a result, the value of the hyperparameter λ can be interpreted as being proportional to the importance of coarse-grained, single-instance features and inversely proportional to the importance of the instance-level features in the classification problem. We later demonstrate that setting λ=0 or λ=1 leads to suboptimal performance, and a value of λ between 0 and 1 gives optimal fusion (discussed further below.

### Predicting High Vs Low PFC Activation During Frustration

We now turn to using our MIF framework to classify individuals with normal vs. low Pre-Frontal Cortex (PFC) neural activation associated with emotion regulation during frustration. Although we collected data from 94 participants, some participants had to be excluded due to technical issues in fNIRS recording (N=13), missing video data (N=1), or missing stimulus recordings to synchronize fNIRS readings with videos (N=4), and we could only use data from 76 participants.

As described above, we extracted ground truth beta values from the fNIRS recordings, which indicate the magnitude of the hemodynamic response during the Negative block as compared to the baseline Rest period. A low beta value is indicative of lower emotion regulation-related neural activity in response to frustration, which is tied to greater psychopathological risk. To split individuals into two groups—individuals with “Normal” vs. “Low” activation levels—we used one standard deviation below the mean activation level across all subjects (mean-1SD) as the threshold, which is a standard practice for psychiatric evaluation (e.g., [59]). This categorization led to 12 individuals classified as exhibiting low activation and 64 individuals with normal levels of activation.

Since our focus is on predicting neural activation during frustration, all features (as described above) were first calculated from the negative feedback segments of the frustration-inducing task. We then calculated the difference between each feature for the negative and positive segments, obtaining twice the number of features we originally had. For the MIL pipeline, each negative feedback segment was considered a separate “instance”. Along with the features extracted from the instance itself, we again calculated the difference between the instance features and the average of each feature across positive feedback segments.

### Baseline Single Instance Learning (SIL) Models

To evaluate the effectiveness of our proposed MIF framework, we first trained baseline SIL models using each of the feature sets described above. We trained and evaluated nine different machine learning models (listed in Appendix C, Table 7) to predict activation levels. Please refer to Appendix C for more details on pre-processing, feature selection, and addressing class imbalance.

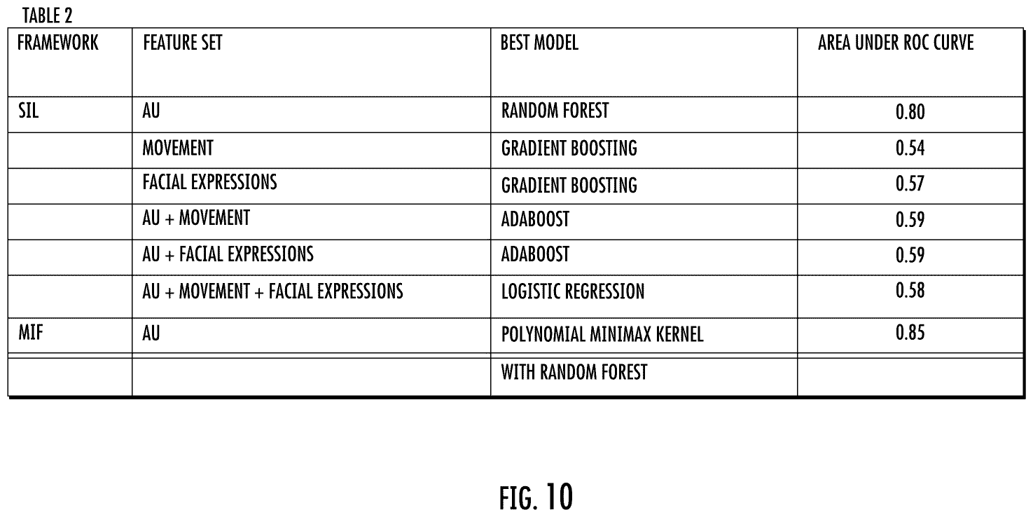

We used a nested cross-validation scheme to select the best hyperparameters for each supervised machine learning algorithm and evaluate the pipeline's generalization performance. We used an outer 5-fold cross-validation scheme that assigns a fifth of all subjects to the test set at each fold, training the model on the
remaining subjects. The test set at each fold is stratified, i.e., each test set contains the same proportion of subjects with normal and low activation. Each subject is only assigned to the test set once. Within the training set at each fold, we applied stratified 3-fold cross-validation to select optimal model hyperparameters. The hyperparameter choices for each algorithm are listed in Table 7. The best performing model in this inner cross-validation loop was evaluated using the test set from the outer fold to obtain an unbiased estimate of classification performance. The best performing SIL algorithm on the outer test sets for each feature set is listed in Table 2, shown in FIG. 10. We find that the AU feature set with a random forest classifier as the supervised learning approach performs best, resulting in an area under the ROC curve (ROC AUC) of 0.80. The importance of AU features in predicting neural activation levels is also supported by previous work in cognitive neuroscience (e.g., intensity of a frowning action has been linked to neural activity in the amygdala and the PFC [64]). It is also interesting to note that logical combinations of AUs into facial expressions have lower predictive power, although these combinations have been found to correlate with neural activity [58]. We hypothesize that the AU feature set contains richer information, since it also includes action units that are not accounted for in the expression combinations (see Appendix A).
   Comparing Multi-scale Instance Fusion Models with SIL Baseline

We further trained and evaluated our proposed Multi-scale Instance Fusion (MIF) framework to classify low vs. normal activation levels. We used the same nested cross-validation scheme detailed above. MIF models were trained using the AU feature set, which resulted in the best performance classifying neural activation in the SIL setting. As part of the bag representation module, we evaluated the seven mapping algorithms discussed in above. As shown in Table 2 (FIG. 10), we found that an MIF model that uses AU features with a Polynomial Minimax Kernel representation achieves the best overall area under ROC curve of 0.85 in classifying PFC activation—a significant improvement over the baseline models. This supports our hypothesis that the MIF framework can improve performance by taking into account instance-level features. We also found that trivial feature aggregation through mean and minimax mappings does not lead to an improvement in performance (ROC AUC=0.80, same as SIL model), demonstrating the utility of formulating the classification as an MIL problem and computing discriminative features at the bag level.

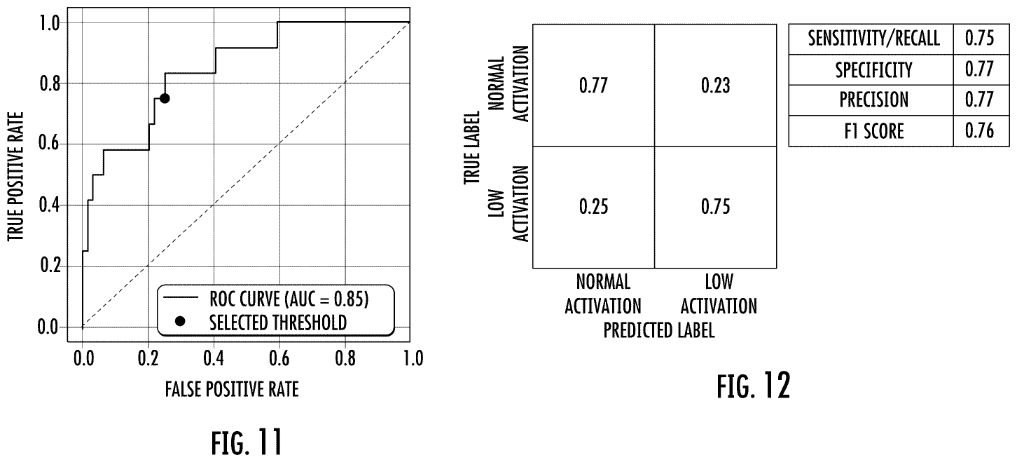

The ROC curve of our MIF model is shown in FIG. 11. The model performance across 5-fold cross validation gave an average area under ROC curve of 0.82 (SD=0.14), suggesting generalizability of the proposed model. We then used the ROC curve to select a probability threshold for classification where the sensitivity and specificity of the model are most balanced (i.e. the threshold minimizing the sensitivity—specificity|metric). This point is shown in FIG. 11—our model achieves a sensitivity of 0.75 and a specificity of 0.77 at the chosen threshold. The normalized confusion matrix showing the fraction of correctly classified individuals in each class is shown in FIG. 12. Note that the threshold can be adjusted to optimize for either of these metrics at the cost of the other—in practice, this decision can be made by experts based on the costs of misidentifying subjects with low and normal activation respectively.

These highly promising results demonstrate the possibility of utilizing EarlyScreen to classify the magnitude of PFC neural activation during frustration using behavioral data. To the best of our knowledge, our work is the first to attempt predicting an objective measure of emotion regulation and to produce proof-of-concept results. Current tools to screen for emotion regulation disorders often depend on symptomatic reports from parents and caregivers, which have been shown to have modest prediction performance. For example, the Child Behaviour Checklist (CBCL) was found to demonstrate a mean sensitivity of 0.66 and specificity of 0.83, while the Strengths and Difficulties Questionnaire (SDQ) achieved a mean sensitivity of 0.49 and specificity of0.93 [128]. Our model is able to achieve sensitivity and specificity levels comparable to these screening tools with less than ten minutes of facial observations that can be collected remotely at home while completing a clinically validated task.

Predicting Psychopathology Diagnosis from Behavioral Features

In addition to identifying objective neural activation levels, we attempted to use automatically extracted facial expressions and movement information to classify the clinical diagnosis status of individuals. As ground truth, we used the children's scores on the CBCL Externalizing subscale, ADHD Inattention and Hyperactivity subscales, and MAP-DB scale as described above to categorize them into those below or above the clinical threshold. Among the 76 children in our study, 25 scored above the threshold on at least one of these scales and were categorized as ‘Clinical’ participants. The other 51 were classified as ‘Non-clinical’ participants, indicating a low risk of psychopathology.

### Baseline Single Instance Learning (SIL) Models

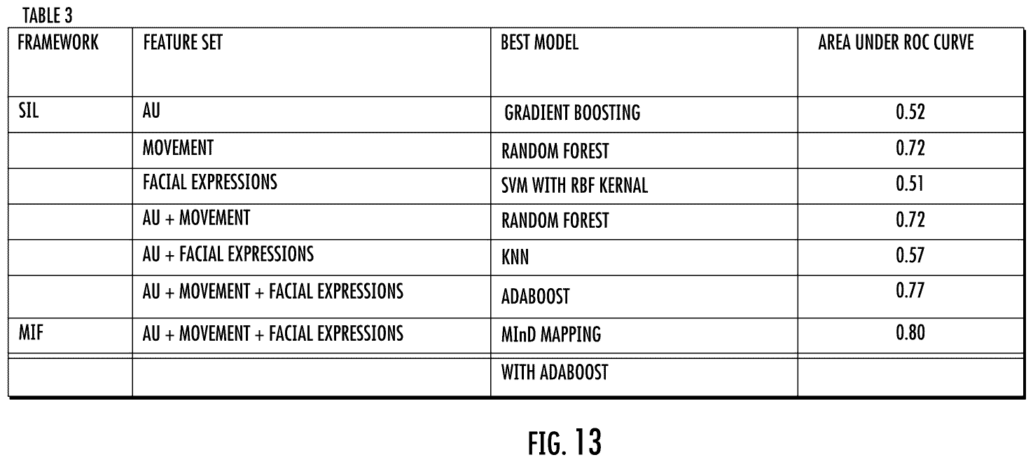

Similar to our pipeline for classifying activation levels, we used a nested cross-validation approach to first train and evaluate baseline SIL models, using the same candidate feature sets described above. Referring to FIG. 13, Table 3 shows the results of our analysis—we found that an AdaBoost classifier using AU+Movement+Facial Expressions features achieves the best classification performance with an ROC AUC of 0.77.

From Table 3 (FIG. 13), we see that Movement features outperform both AU and Facial Expressions separately at predicting clinical scores. Prior work has also shown associations between eye and body movements and scores on psychopathological scales. Children with higher scores on the CBCL and other diagnostic scales have been found to exhibit higher bodily movements [7], and studies have identified differences in eye movements and gaze patterns among ADHD and control subjects [83]. We found that the addition of AU and Facial Expressions to the Movement feature set resulted in further improvement in classification performance, which is supported by prior work on facial expressions [41].

Comparing Multi-scale Instance Fusion Models with SIL Baseline

We then investigated whether our proposed MIF framework improves classification performance by combining information from representations of instance-level features. Similar to the procedure described for classifying neural activation, we trained MIF models using the AU+Movement+Facial Expressions feature set and an AdaBoost classifier within the supervised learning module.

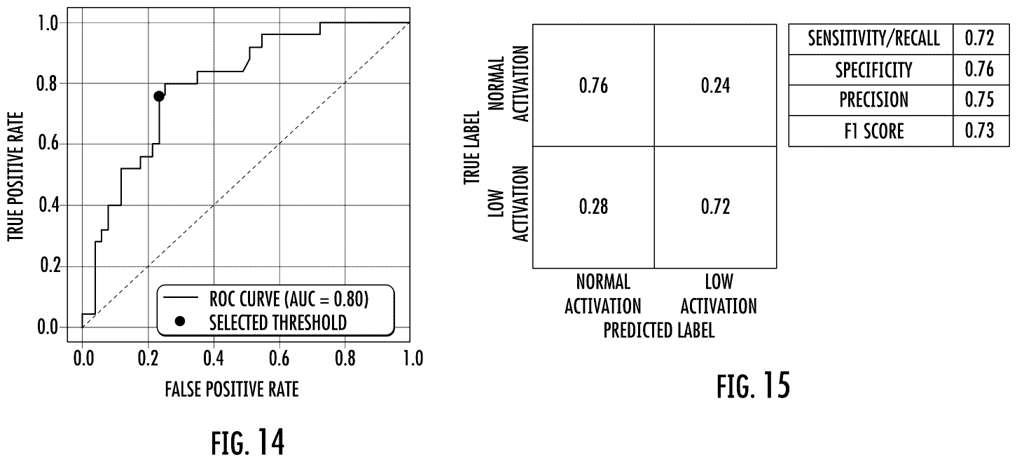

We found that an MIF model with MInD bag representation achieved the best overall area under ROC curve of using AU+Movement+Facial Expressions, compared to an AUC of 0.77 in the baseline SIL setting. Similar to neural activation classification, we found that MInD mapping, which accounts for dissimilarities between bags, improves performance, whereas statistical aggregation methods do not. The mean test area under ROC curve of our best performing model over 5 folds was 0.79 (SD=0.05). We then selected a threshold for classification based on the ROC curve to minimize the |sensitivity—specificity|metric. This point is shown in FIG. 14—the model achieves a sensitivity of 0.72 and a specificity of 0.76 at this threshold. The normalized confusion matrix depicting the performance of the classifier is shown in FIG. 15.

The performance of our model is comparable to recent approaches that predict clinical symptoms using behavioral data. For instance, Place et al. predict depressive mood using mobile sensing with an area under ROC curve of 0.74 [104]. Mock et al. classify individuals scoring high vs. low (top and bottom third of scores—not clinical vs. non-clinical risk status) on ADHD inattention and hyperactivity subscales with an accuracy of 81.1% and 88.9% using touch interaction features [94]. Our approach performs reasonably well at the difficult problem of categorizing psychopathological risk status in preschool-aged children using a short and unobtrusive behavioral screening tool. This supports the applicability of EarlyScreen as a screening procedure for emotion regulation-related psychopathological disorders in the wild.

Association between Model Predictions and Psychometric Scores

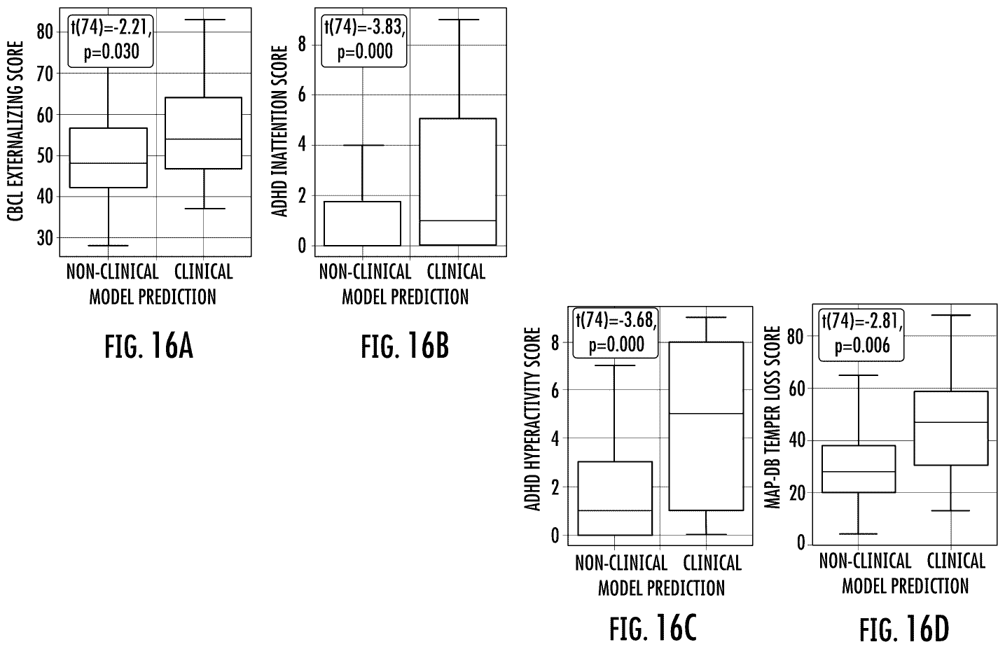

To further test the validity of our psychopathology classification model, we compared the distribution of reference or ground truth scores on the CBCL Externalizing Behavior, ADHD Inattention, ADHD Hyperactivity, and MAP-DB Temper Loss subscales among the individuals predicted to be Clinical and Non-Clinical. FIGS. 16A-16D show the distribution of the scores for each predicted class, respectively. The mean scores of the subjects predicted to be Clinical were significantly higher than those of the subjects predicted to be Non-Clinical on all psychometric scales (p<0.05).

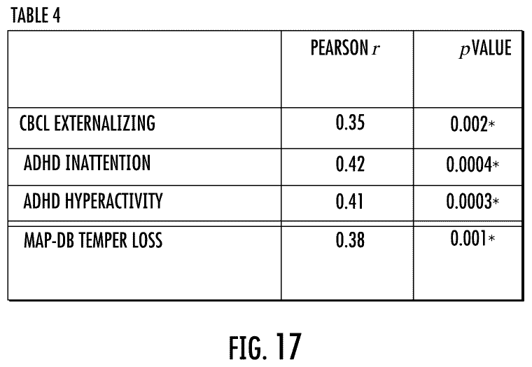

We also examined the association between the model's predicted probability of classifying an individual as Clinical and their scores on the psychopathology scales (see Table 4, shown in FIG. 17). We found a statistically significant positive correlation between predicted probability of being above Clinical threshold and the participants' score on each psychometric subscale (p<0.01 for all subscales). Since our analysis involves multiple comparisons, we used the Benjamini-Hochberg procedure [19] to control for the false discovery rate (FDR) at significance level α=0.05. All t-tests and correlations reported above remain significant after FDR adjustment. Overall, our analysis indicates that the predictive model is consistent with real-world diagnostic tools at identifying individuals with significant psychopathological risk.

### Optimal Bag Representation and Information Fusion

### Bag Representation

As part of our analysis, we also evaluated two statistical feature aggregation methods—Mean and Minimax mapping—as baselines for the Bag Representation Module (see above) in the MIF framework. Notably, we found no improvement using these statistical aggregates as the bag representations in either classification problem. In both cases, the MIF models with Mean and Minimax representation achieved the same ROC AUC as the SIL baselines, underscoring the ineffectiveness of aggregation-based bag representations.

We instead found that the Polynomial Minimax Kernel representation outperformed other approaches in classifying neural activation levels, while MInD mapping achieved the highest ROC AUC in classifying psychopathological disorder status. Both these bag representation algorithms account for overall bag-to-bag similarities, as opposed to other MIL mappings which compute bag-to-instance similarities. This suggests that inter-individual differences in emotion regulation responses outweigh differences between trial-level responses across participants.

### Information Fusion

As discussed above, the information fusion approach in the MIF framework guarantees that the MIF model will perform at least as well as the best SIL and MIL pipelines that constitute it. The results above indicate that there is a significant improvement in classification performance for both neural activation and psychopathology detection when the SIL pipeline is augmented as proposed in our framework.

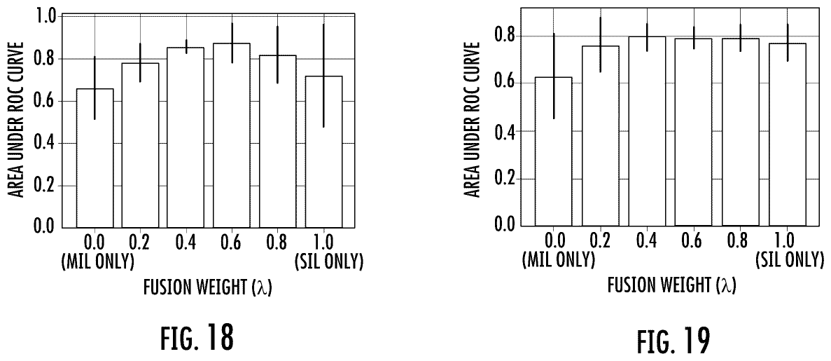

FIG. 18 shows the performance of the proposed MIF model for predicting neural activation using different values of λ for information fusion. As described above, λ=0 corresponds to using only the MIL pipeline for predictions, while setting λ=1 leads to using only the SIL pipeline. Any value of λ between 0 and 1 is a fusion of both pipelines. We observed that setting λ=0.6 achieves the best classification performance (mean=0.87, SD=0.09) for neural activation prediction. FIG. 19 shows the average ROC AUC for different values of λ for the MIF model to predict clinical disorder status. The best performing model achieved a mean AUC of 0.79 (SD=0.06) at λ=0.4. We found that a fusion architecture performed better than both a standalone SIL pipeline and a standalone MIL pipeline. This is true for both our neural activation and clinical status prediction models, and underscores the utility of combining multi-scale feature representations for such prediction problems.

### Deployment Considerations

### Fairness and Ethical Considerations

We now discuss ethical considerations related to deploying EarlyScreen's neural activation or psychopathology classifiers in the real world. We ground this discussion in the model reporting criteria proposed by Mitchell et al. [93], providing a comprehensive review of the intended use, relevant factors, and metrics related to our models.

Ethical Considerations: As described above, a primary aim of the work was to investigate whether facial expressions and movement-related behavioral data automatically extracted from facial videos can be utilized to predict coarse-grained neural activation during frustration. In doing so, we used facial AUs extracted using computer vision algorithms and leveraged their known association with neural activity [58]. It is important to note that we do not attempt to predict emotion using these facial AUs—there is significant debate about the validity of using facial movements to infer emotions (see [15]). The use of gamified tasks to induce frustration has also been validated previously [59].

Additionally, since our model uses data extracted from video recordings of children's faces, it is important to consider the privacy implications of collecting and processing this data in a real-world deployment. This is especially important since our models act as mental health screening tools, whose outcomes might lead to undue stress or stigmatization if not handled correctly [63]. To minimize unintended harms and biases, the models do not use any demographic data or protected information to make predictions.

Intended use: As part of EarlyScreen, we present two MIF models trained on facial AUs and movement-related features that are each intended to be used for the following purposes, respectively:
As a screening tool for identifying preschool-aged children who may exhibit low neural activation during frustration, a risk factor for broad psychopathology.
As a screening tool for identifying preschool-aged children who fall above the clinical threshold for specific disorders common in early childhood.

The proposed models are intended to be used as screening tools that could assist a childhood mental health practitioner in making a rapid and accurate diagnosis. In addition, the models are not meant to be used to detect faces or facial characteristics, or infer felt emotions or personal characteristics of users.

Factors Affecting Model Performance: As with other human-centered computer vision technology, our models' performance is likely to be impacted by several factors related to participants' identity and personal characteristics such as gender, age, race and ethnicity, Fitzpatrick skin type [53] etc. It might also be affected by complex interactions of these features, as well as external factors such as camera hardware and placement, lighting, other environmental factors, etc. Note that this is in addition to individual differences in emotion regulation itself, which may be influenced by factors outside the purview of this work.

We present quantitative analysis of the performance of our prediction models disaggregated by the demographic factors available in our dataset, i.e., gender, age group, and race. For a breakdown of our subject population as well as the number and percentage of participants with low neural activation or clinical symptoms in each demographic subgroup, please refer to Appendix D. We examine model performance with respect to each unitary factor.

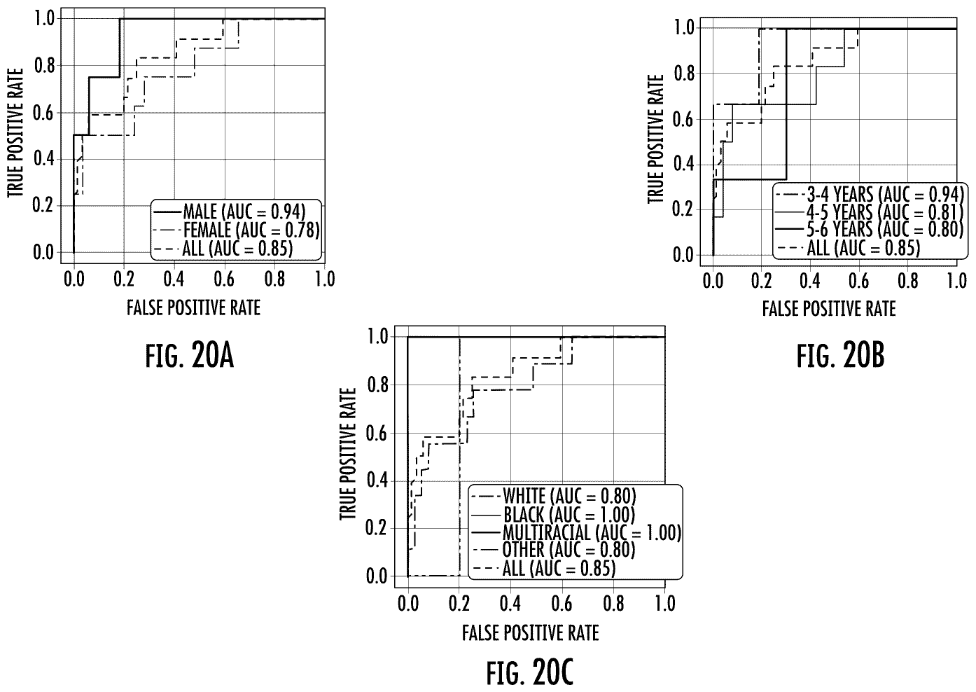

We first evaluate the performance of our neural activation prediction model by demographic group. FIGS. 20A-20C show the ROC curves for each subgroup split by gender, age, and race along with the overall ROC curve. We find that the model achieves a higher ROC AUC for males (0.94) than for females (0.78). The model performance is also higher among younger participants—the AUC for 3 to 4-year-olds is 0.94 while that for ages 4-5 and 5-6 are 0.81 and 0.80 respectively. Disaggregating by racial groups, we see that the model achieves an AUC of 1.0 for Black and Multiracial participants and an AUC of 0.80 among participants self-identifying as White or Other racial group.

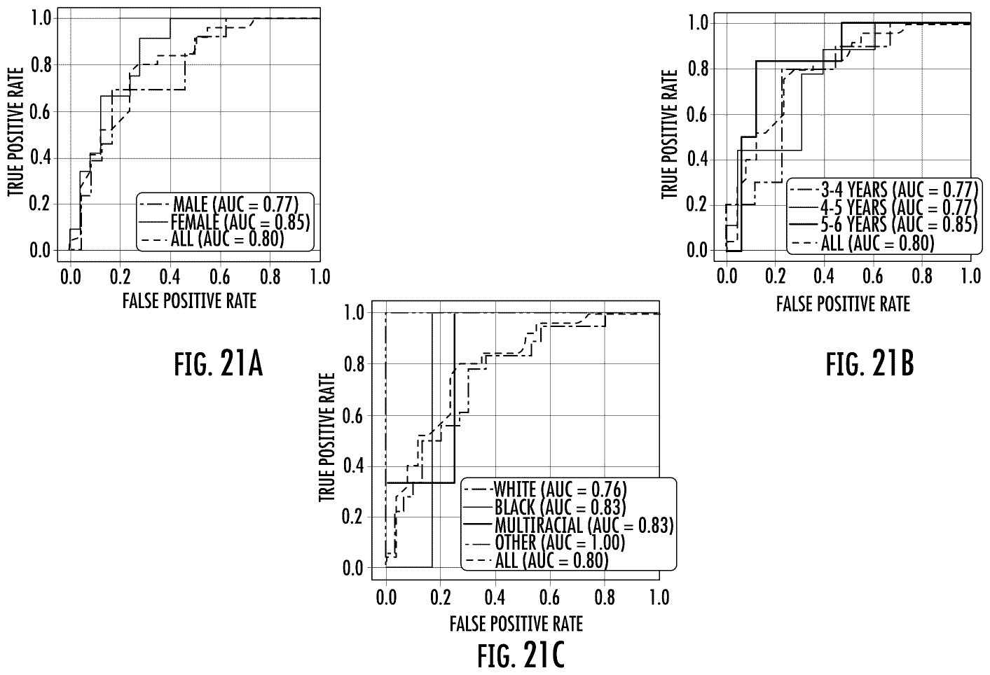

Separating demographic subgroups and evaluating the performance of our clinical psychopathology classification model, we find that the ROC AUC for male participants is 0.77 and that for female participants is 0.85 (see FIGS. 21A-21C). Across age groups, we see that the model achieves an ROC AUC of 0.77 among ages 3-4 and 4-5, while the performance among 5- to 6-year-olds is slightly higher at 0.85. We also observe that the model exhibits an ROC AUC of 0.76 among White participants, 0.83 among Black as well as among multiracial participants, and 1.0 among participants from Other racial groups.

The above analysis shows that our models achieve reasonable performance across different demographic subgroups. Although the performance of the model varies with the number of participants in each subgroup and the base rate of abnormalities in the subgroups, the relative consistency of the results is encouraging for the deployment of these models in the real world. Further research should validate their performance in broader populations.

### Utility for Practitioners

To evaluate the usability, utility, and drawbacks of EarlyScreen from a clinical perspective, we conducted a user survey of mental health professionals who were introduced to this technology and asked for feedback that could be incorporated into future versions. Practitioners were recruited through the American Psychological Association's mailing list for the Society of Clinical Child and Adolescent Psychology and were offered a $5 gift card for their participation. A total of 60 respondents completed the survey, of which 11 completed it partially. We present preliminary findings from this survey to illustrate how EarlyScreen can support current clinical practices.

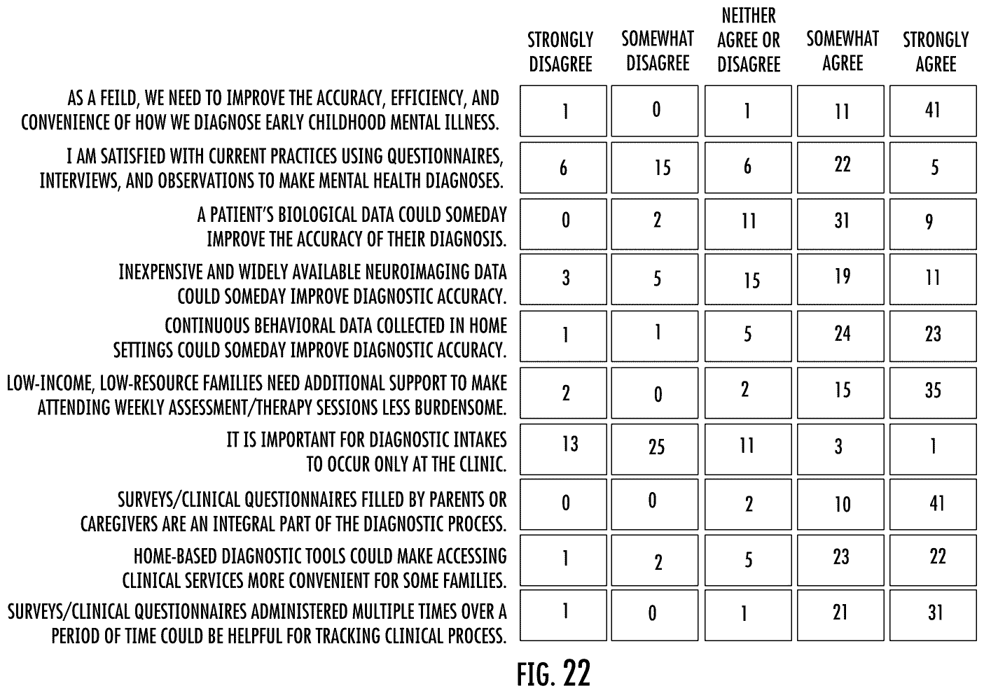

Need for Home-Based Diagnostic Tools and Novel Data Sources: Participants were first asked about their current clinical practices, their satisfaction with existing diagnostic measures, and any additional sources of diagnostic information they would like to access. They were not introduced to this research or EarlyScreen at this stage to obtain unbiased responses about their actual clinical needs and attitudes about at-home care. FIG. 22 shows the number of respondents who rated each statement between 1 (“Strongly disagree”) and 5 (“Strongly agree”). An overwhelming majority of respondents strongly agreed (N=41 or 75.9%) or somewhat agreed (N=11 or 20.4%) that As afield, we need to improve the accuracy, efficiency, and convenience of how we diagnose early childhood mental illness. Most of the respondents felt that patients' biological (N=40 or 75.5%), behavioral (N=47 or 87%), and neuroimaging data (V=30 or 56.6%) could someday improve diagnostic accuracy, and that questionnaires filled by parents or caregivers are an integral part of the diagnostic process (N=51 or 96.2%). These responses indicate the potential clinical utility of both the behavioral videos and the subsequent metrics predicted by EarlyScreen.

Clinicians also showed positive attitudes towards at-home screening, with 38 respondents (71.7%) disagreeing with the idea that it is important for diagnostic intakes to occur only at the clinic. Nearly all respondents (N=50 or 92.6%) also felt that low-income, low-resource families need additional support to make attending weekly assessment therapy sessions less burdensome, and that home-based diagnostic tools could make accessing clinical services more convenient for some families (N=45 or 84.9%). EarlyScreen is a step towards realizing this vision of convenient, at-home mental health screening through accessible mobile applications.

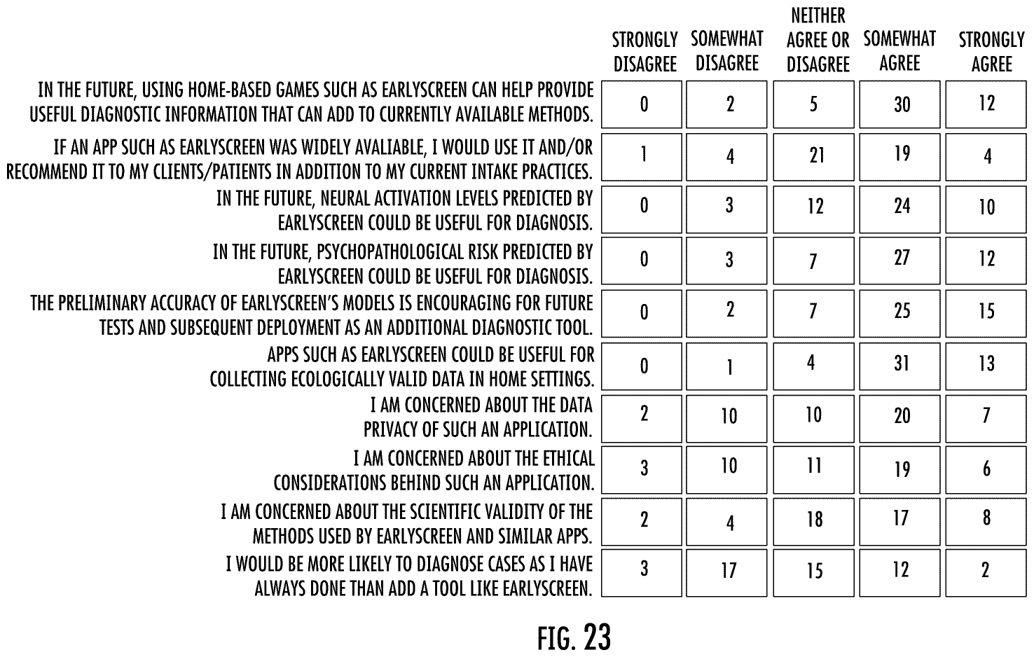

Diagnostic Utility and Performance of Earlyscreen: Participants were then presented with a description of the EarlyScreen prototype presented in this paper, including the predicted metrics, behavioral features used in the predictive models, and classification performance for both neural activation and psychopathology prediction. Based on this description, they were asked to rate their agreement with a series of statements relating to the diagnostic utility of EarlyScreen and its predicted outcomes and their willingness to incorporate such applications into their current practice. FIG. 23 shows the participants' aggregate feedback on a scale of 1 (“Strongly disagree”) to 5 (“Strongly agree”). Additionally, respondents also had the opportunity to comment on what they liked or disliked the most about EarlyScreen, what features they would like to see in a future version, and elaborate on any potential concerns they had. These comments are quoted here along with the participant ID.

Most of the respondents (N=42 or 85.7%) agreed that using home-based games such as EarlyScreen can help provide useful diagnostic information that can add to currently available methods. Clinicians especially noted that such tools would “increase access to care for underserved communities” (P3) and “reduce the wait-list time” (P15) for accessing clinical services.

44 respondents (89.8%) said that such applications could be useful for collecting ecologically valid data in home settings. They also noted the advantages of EarlyScreen using a “child-friendly format” (P10) such that “it would be easy to engage children in using [EarlyScreen] and would provide complementary information to a traditional intake process” (P11). P24 also mentioned the utility of having access to “real-time information at home”, while P38 noted the “passive ability to gather information”.

Participants also felt that neural activation levels (N=34 or 69.4%) and psychopathological risk (N=39 or 79.6%) predicted by EarlyScreen could be useful for diagnosis in the future. P15 noted that EarlyScreen “can capture valuable data about brain activity that's not now available—and in a format that kids will easily engage with—data that's so relevant for early detection of developmental issues where early intervention matters so much in terms of outcome!”.

40 participants (81.6%) also responded that the preliminary accuracy of EarlyScreen's models is encouraging for future tests and subsequent deployment as an additional diagnostic tool, with “higher percentage predictive value compared to behavior checklists” (P16).

23 respondents (46.9%) said they would use and/or recommend an app such as EarlyScreen to clients/patients in addition to current intake practices, while 21 others (42.9%) expressed a neutral opinion. 20 respondents (40.8%) disagreed that they would be more likely to diagnose cases as [they] have always done than add a tool like EarlyScreen, while 15 or 30.6% were neutral. The respondents elaborated on their reasons further, for example, P7 pointed out that “there would need to be explicit guidance on how to integrate info from the app with other assessments”, P2 noted the presence of “institutional barriers and them not being flexible to introducing new techniques”, and P31 was “concerned about startup costs and training”.

Overall, clinicians demonstrated positive attitudes about EarlyScreen's utility and performance. Responses about using or recommending EarlyScreen also largely positive, and participants identified training resources that should be integrated into future versions for wider adoption.

Concerns for Deployment: Practitioners in our study were also asked about potential concerns related to the deployment of apps such as EarlyScreen. 27 participants (55.1%) expressed concerns about data privacy, wanting to “know how the data is stored” (P22) as well as information “on who develops, maintains, or monitors the app” (P7). Respondents also expressed concerns about ethical considerations (N=25 or 51%) behind applications such as EarlyScreen. They noted a “concern about over reliance on something like this rather than a more full clinical assessment” (P29), which is in line with previous research on clinician attitudes towards other tools such as standardized assessments [74]. P27 pointed out that “facial recognition needs to be validated across physical characteristics . . . it should be validated across groups”, which we partially address above. Participants were also concerned about “the lack of consideration of child's environmental context in the assessment process” (P37) and “the need to standardize testing/assessment environment somewhat across homes (e.g., minimize potential distractions from siblings, etc.)” (P33). Another concern was “about accessibility for families with limited access to high speed internet or smartphones” (P10). The scientific validity of the methods used by EarlyScreen and similar apps was also noted as a concern (N=25 or 5.1%), with clinicians noting that “it still needs more validation” (P11) but also that they “want to understand how it was developed, read more about the validity and reliability” (P44). This can be explained by the lack of details in the overview of EarlyScreen that was provided, with participants noting the need for published research in a “peer-reviewed journal explaining those processes, explaining methods, etc. Once that body of literature was established and I had a lot more information, my response would be on the upper end of the scale” (P27).

In summary, practitioners emphasized the need to ensure that data protection policies are in place and that standardization measures are taken while deploying EarlyScreen. In addition, follow-up work should examine the performance of the models in a broader population and further test the reliability and validity of EarlyScreen prior to its deployment in the wild.

### Considerations for Mobile Deployment

In addition to ensuring ethical usage and consistent performance across demographic subgroups, EarlyScreen must be robust against various environmental and device-related factors for successful deployment in home settings. One such consideration is noisy video capture or the occlusion of children's faces during the task due to head movement or fidgeting, camera occlusion, or device positioning. To account for these factors, children in this study were not asked to maintain a fixed seating position or posture, and were free to move around in the seat, look around, talk to the experimenter, and behave as they would in an unsupervised setting at home.

Furthermore, we examined the amount of movement exhibited by our participants during the feedback segments of the frustration-inducing task. We found that the children moved their head a mean distance of 341.7 centimeters per second relative to the fixed camera during the feedback segments on average, implying that our system is robust to a fair degree of human movement. We also use head pose and gaze direction as features in our models, enabling us to leverage inter-individual variability in movement to predict neural activation and psychopathology. Our system is also trained on data with some degree of facial occlusion due to movement and camera angle—35.6% of the frames captured during positive or negative feedback had missing faces (or facial landmarks could not be captured with high confidence).

In addition to allowing for noise in the video capture process, our experimental conditions also emulate at-home gameplay on a tablet by using a stationary touchscreen monitor for the kids to interact with. The cameras used for data collection (Axis Communications PTZ Network Cameras) capture video at a resolution of 1080p and frame rate of 60 Hz, which is easily achieved by current smartphone and tablet cameras.

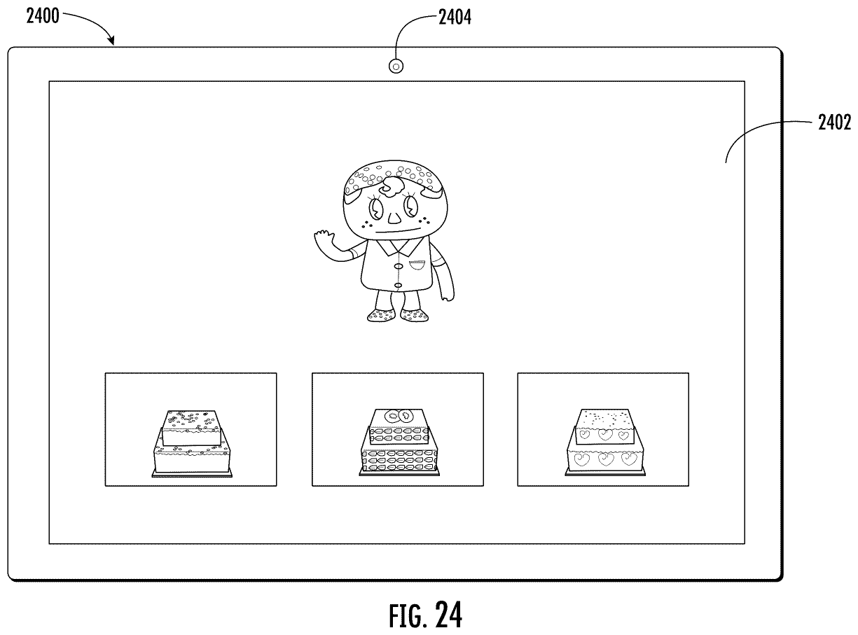

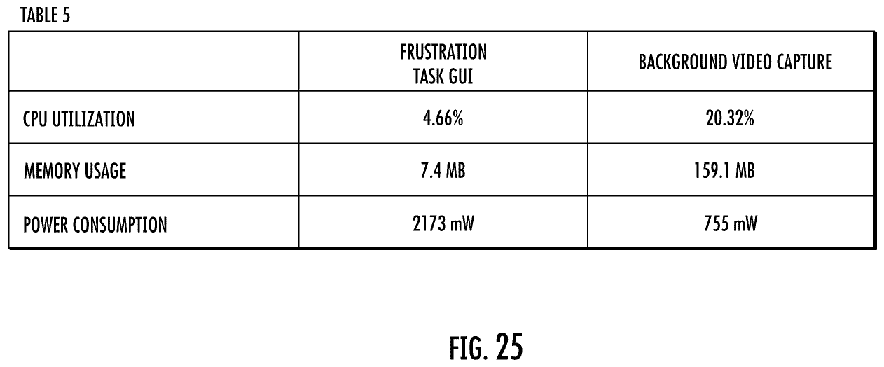

We also implemented a pilot version of the frustration-inducing task on a Windows Surface Pro 6 tablet 2400 as shown in FIG. 24. The touchscreen 2402 tablet 2400 runs the Windows 10 Pro operating system with an Intel Core i5 processor and 8 GB of installed RAM, and has a 5 MP front-facing camera 2404 that can capture 1080p HD video. OBS Studio was used for background video capture from the front-facing camera 2404 throughout the duration of the task. We profiled the performance of our task implementation on this device and report detailed statistics in Table 5, shown in FIG. 25. Our analysis shows that EarlyScreen can be easily deployed on existing commercial devices for use in a child's home. A larger-scale pilot study is currently underway to validate the usability and screening accuracy of the tablet application.

### DISCUSSION

We now discuss the implications of this work for both mental health practitioners and UbiComp researchers. We also discuss the fairness and ethical considerations of deploying EarlyScreen in the real world, describe its limitations, and delineate some future research directions.

### Implications for Clinical Psychology

The results of the present study have clear implications for the diagnosis of mental disorders in early childhood. Identifying mental disorder in children as young as preschoolers is very difficult as the symptoms of disorder closely resemble normative misbehavior [123] and many diagnostic instruments commonly used in real-world clinical settings have AUCs in the 0.7 range [21, 69]. Moreover, accessing these diagnostic services often places a significant burden on parents [24] and millions of children go undiagnosed and untreated each year [131]. To our knowledge, this study was the first to attempt to classify disorder status from standard streaming video. The fact that this initial effort achieved similar accuracy to instruments commonly used in the field, using automated methods that could be administered at home suggests the exciting possibility that ubiquitous computing methods could improve the accuracy of and reduce barriers to obtaining an early diagnosis. As noted by the mental health professionals surveyed, EarlyScreen provides the “ability to collect behavioral information in real world setting” (P26) in a way that “feels natural to the child and not like an evaluation” (P27) and “doesn't need connection to official medical services to use” (P44).

Moreover, EarlyScreen showed not only surprisingly good detection of disorder status, but detection of individual differences in PFC activation during frustration, a key neural mechanism that drives the development of mental illness [32, 36, 78]. Building on this finding may facilitate the development of diagnostic tools that allow clinicians to account for neural activation in their clinical decision making, providing “valuable data about brain activity that's not now available” (P15). Further, results suggest it is possible to infer coarse-grained PFC activation during emotion regulation using our proposed models with any mobile platform with a front facing camera, without requiring specialized neuroimaging hardware. Future research may therefore be able to study the early development neural underpinnings of early childhood mental health disorders at a much larger scale than is currently possible.

### Implications for UbiComp Research

There is burgeoning interest among UbiComp researchers studying mental health to measure neural, physiological, and behavioral markers of mental disorders. Researchers have recently shown that resting state functional connectivity between the subgenual cingulate cortex and the ventromedial/orbitofrontal cortex is correlated with smartphone screen time [68] and that smartphone usage can predict functional connectivity [98] between the ventromedial prefrontal cortex and the amygdala—both of which have important implications for diagnosing depression and anxiety-related disorders. EarlyScreen further demonstrates how sensing tools can leverage the association between neural and behavioral responses to support the diagnosis of psychopathology related to emotion dysregulation.

We also introduce a multi-trial frustration-inducing task with baseline, positive, and negative feedback periods that can be easily deployed as a gamified smartphone application. The task (or “game”) is self-contained, which means that parents or caregivers can simply download the application and let children “play” it without additional setup or clinical intervention. The application can then utilize the device's front-facing camera to assess children's risk in real time and share the results with a caregiver, teacher, or clinician. A pilot implementation of such an application is described above. EarlyScreen can also be used to collect contextual information about children's behavior at home, allowing mental health practitioners to track children's progress or response to clinical interventions over time without requiring additional clinical visits. This would lead to more ecologically valid data for diagnosis compared to current methods of in-clinic behavior observation sessions.

While EarlyScreen utilizes vision-based techniques for monitoring neural activity during naturalistic instances of emotion regulation, there is also potential for using wearable devices for this purpose. There has been work in the UbiComp community on the detection of upper facial AUs using eyeglass-type wearables [107]. Movement, head pose, and eye gaze can also be detected using a variety of wearable devices with cameras and inertial sensors (e.g., [31, 66, 134]). These devices can potentially be used to continuously monitor AUs and other facial features and to detect neural activation levels in the wild. Further research is required to determine the accuracy of such systems trained with a subset of features and in more challenging contexts.

Our work also introduces a novel machine learning framework based on multi-scale instance fusion. This architecture can be adapted and used for both classification and regression problems in a number of domains beyond mental health applications. Drawing on core ideas from ensemble learning and multiple instance learning, we believe that our framework can be used to improve prediction performance in scenarios where the experimental design involves multiple trials, including but not limited to longitudinal data collected over multiple sessions or days. It can also be useful for prediction problems with audio, video, or image data where features extracted at multiple spatio-temporal resolutions can be combined in a similar manner. Such experimental paradigms are quite common in UbiComp research, and we believe that our framework will be a useful tool for researchers who encounter these scenarios in their work.

### Limitations and Future Work

Our work is an exploratory proof of concept for predicting neural activation and clinical diagnostic status using facial expressions and movement-related features, and thus has limitations that will be addressed in future research. EarlyScreen depends on OpenFace 2.0 to extract AUs accurately, and extracted AUs have not been compared with manual FACS coding to verify their accuracy. As discussed previously, preschool-aged children also tend to look around, cover their faces with their hands, or otherwise move in a way that their faces are partially occluded from the camera's field of view at times. These could cause errors in AU recognition—however, OpenFace reports an average concordance coefficient of 0.73 using a baseline validation dataset [11].

Another limitation of our work is the relatively small sample size (N=76) in training our models, however, our 5-fold cross-validation results and demographic analysis are encouraging for real-world deployment. To further assess the real-world generalizability of our system, we are also in the process of conducting at-home validation studies using the tablet-based implementation of our clinically-validated frustration-inducing task. Following this, we aim to engage diverse stakeholders including parents, caregivers, and child psychologists in a human-centered design process to create the final implementation of EarlyScreen. Our survey of clinical practitioners is a step in this direction, allowing us to discover the concerns they have about the future deployment of EarlyScreen.

### CONCLUSION

In this work, we presented EarlyScreen, a system utilizing facial expressions and movement-related features from videos to characterize emotion regulation during frustration in preschool-aged children. We conducted an exploratory study with 94 participants where we recorded facial videos as well as neural activation using fNIRS while the children were engaged in a frustration-inducing task. We first attempted to classify low vs. normal activation levels in the PFC using features extracted from facial action units exhibited during positive and negative feedback. We trained and evaluated a novel machine learning framework, the Multi-scale Instance Fusion (MIF) framework, to classify activation levels. Our model succeeded in classifying PFC activation with an area under the ROC curve of 0.85. Next, we showed that behavioral features could also be used to predict psychopathology diagnosis using our MIF framework, achieving an area under the ROC curve of 0.80. The performance of EarlyScreen is on par with that of widely-used clinical assessment tools and consistent with individuals' scores on clinically-validated psychometric evaluation scales. Furthermore, we received positive feedback on the clinical utility of EarlyScreen from a survey of 60 child mental health professionals. We hope that our work is a step towards developing behavioral sensing solutions to better understand the neural underpinnings of psychopathology.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

### REFERENCES

All references are incorporated herein by reference in their entirety.
[1] Saeed Abdullah and Tanzeem Choudhury. 2018. Sensing technologies for monitoring serious mental illnesses. IEEE MultiMedia 25, 1 (2018), 61-75.
[2] Saeed Abdullah, Mark Matthews, Ellen Frank, Gavin Doherty, Geri Gay, and Tanzeem Choudhury. 2016. Automatic detection of social rhythms in bipolar disorder. Journal of the American Medical Informatics Association 23, 3 (2016), 538-543.
[3] Thomas M Achenbach and Leslie A Rescorla. 2000. Manual for the ASEBA preschool forms and profiles. Vol. 30. Burlington, VT: University of Vermont, Research center for children, youth, & families.
[4] Nadav Aharony, Wei Pan, Cory Ip, Inas Khayal, and Alex Pentland. 2011. Social fMRI: Investigating and shaping social mechanisms in the real world. Pervasive and Mobile Computing 7, 6 (2011), 643-659.
[5] Mawulolo K Ameko, Miranda L Beltzer, Lihua Cai, Mehdi Boukhechba, Bethany A Teachman, and Laura E Barnes. 2020. Offline contextual multi-armed bandits for mobile health interventions: A case study on emotion regulation. In Fourteenth ACM Conference on Recommender Systems. 249-258.
[6] Stuart Andrews, Ioannis Tsochantaridis, and Thomas Hofmann. 2002. Support Vector Machines for Multiple-Instance Learning. In NIPS, Vol. 2. Citeseer, 561-568.
[7] Inge Antrop, Herbert Roeyers, Paulette Van Oost, and Ann Buysse. 2000. Stimulation seeking and hyperactivity in children with ADHD. Journal of Child Psychology and Psychiatry 41, 2 (2000), 225-231.
[8] Shelli Avenevoli, Joseph C Blader, and Ellen Leibenluft. 2015. Irritability in Youth: An Update. Journal of the American Academy of Child and Adolescent Psychiatry 54, 11 (2015), 881.
[9] Roger Azevedo, Michelle Taub, Nicholas V Mudrick, Garrett C Millar, Amanda E Bradbury, and Megan J Price. 2017. Using data visualizations to foster emotion regulation during self-regulated learning with advanced learning technologies. In Informational environments. Springer, 225-247.
[10] Tadas Baltrušaitis, Marwa Mahmoud, and Peter Robinson. 2015. Cross-dataset learning and person-specific normalisation for automatic action unit detection. In 2015 11th IEEE International Conference and Workshops on Automatic Face and Gesture Recognition (FG), Vol. 6. IEEE, 1-6.
[11] Tadas Baltrusaitis, Amir Zadeh, Yao Chong Lim, and Louis-Philippe Morency. 2018. Openface 2.0: Facial behavior analysis toolkit. In
2018 13th IEEE International Conference on Automatic Face & Gesture Recognition (FG 2018). IEEE, 59-66.
[12] Jeffrey W Barker, Ardalan Aarabi, and Theodore J Huppert. 2013. Autoregressive model based algorithm for correcting motion and serially correlated errors in fNIRS. Biomedical optics express 4, 8 (2013), 1366-1379.
[13] Russell A Barkley. 1997. ADHD and the nature of self-control. Guilford Press.
[14] Ian Barnett, John Torous, Patrick Staples, Luis Sandoval, Matcheri Keshavan, and Jukka-Pekka Onnela. 2018. Relapse prediction in schizophrenia through digital phenotyping: a pilot study. Neuropsychopharmacology 43, 8 (2018), 1660-1666.
[15] Lisa Feldman Barrett, Ralph Adolphs, Stacy Marsella, Aleix M Martinez, and Seth D Pollak. 2019. Emotional expressions reconsidered: Challenges to inferring emotion from human facial movements. Psychological science in the public interest 20, 1 (2019), 1-68.
[16] Jennifer Beecham. 2014. Annual research review: child and adolescent mental health interventions: a review of progress in economic studies across different disorders. Journal of Child Psychology and Psychiatry 55, 6 (2014), 714-732.
[17] Ashkan Beheshti, Mira-Lynn Chavanon, and Hanna Christiansen. 2020. Emotion dysregulation in adults with attention deficit hyperactivity disorder: a meta-analysis. BMC psychiatry 20, 1 (2020), 1-11.
[18] Dror Ben-Zeev, Christopher J Brenner, Mark Begale, Jennifer Duffecy, David C Mohr, and Kim T Mueser. 2014. Feasibility, acceptability, and preliminary efficacy of a smartphone intervention for schizophrenia. Schizophrenia bulletin 40, 6 (2014), 1244-1253.
[19] Yoav Benjamini and Yosef Hochberg. 1995. Controlling the false discovery rate: a practical and powerful approach to multiple testing. Journal of the Royal statistical society: series B (Methodological) 57, 1 (1995), 289-300.
[20] Vinay Bettadapura. 2012. Face expression recognition and analysis: the state of the art. arXiv preprint arXiv:1203.6722 (2012).
[21] J Biederman, MC Monuteaux, E Kendrick, KL Klein, and SV Faraone. 2005. The CBCL as a screen for psychiatric comorbidity in paediatric patients with ADHD. Archives of Disease in Childhood 90, 10 (2005), 1010-1015.
[22] Robert J R Blair. 2016. The neurobiology of impulsive aggression. Journal of child and adolescent psychopharmacology 26, 1 (2016), 4-9.
[23] Tibor Bosse and Frank P J De Lange. 2008. Estimating emotion regulation capabilities. In Proceedings of the 1st international conference on PErvasive Technologies Related to Assistive Environments. 1-8.
[24] Elizabeth H Bringewatt and Elizabeth T Gershoff. 2010. Falling through the cracks: Gaps and barriers in the mental health system for America's disadvantaged children. Children and Youth Services Review 32, 10 (2010), 1291-1299.
[25] Rebecca A Burwell and Stephen R Shirk. 2007. Subtypes of rumination in adolescence: Associations between brooding, reflection, depressive symptoms, and coping. Journal of Clinical Child and Adolescent Psychology 36, 1 (2007), 56-65.
[26] Susan D Calkins, Susan E Dedmon, Kathryn L Gill, Laura E Lomax, and Laura M Johnson. 2002. Frustration in infancy: Implications for emotion regulation, physiological processes, and temperament. Infancy 3, 2 (2002), 175-197.
[27] Luca Canzian and Mirco Musolesi. 2015. Trajectories of depression: unobtrusive monitoring of depressive states by means of smartphone mobility traces analysis. In Proceedings of the 2015 ACM international joint conference on pervasive and ubiquitous computing. 1293-1304.
[28] Marc-André Carbonneau, Veronika Cheplygina, Eric Granger, and Ghyslain Gagnon. 2018. Multiple instance learning: A survey of problem characteristics and applications. Pattern Recognition 77 (2018), 329-353.
[29] Yixin Chen, Jinbo Bi, and James Ze Wang. 2006. MILES: Multiple-instance learning via embedded instance selection. IEEE Transactions on Pattern Analysis and Machine Intelligence 28, 12 (2006), 1931-1947.
[30] Veronika Cheplygina, David M J Tax, and Marco Loog. 2015. Multiple instance learning with bag dissimilarities. Pattern recognition 48, 1 (2015), 264-275.
[31] Chul Woo Cho, Ji Woo Lee, Kwang Yong Shin, Eui Chul Lee, Kang Ryoung Park, Heekyung Lee, and Jihun Cha. 2012. Gaze Detection by Wearable Eye-Tracking and NIR LED-Based Head-Tracking Device Based on SVR. Etri Journal 34, 4 (2012), 542-552.
[32] Emil F Coccaro, Chandra Sekhar Sripada, Rachel N Yanowitch, and K Luan Phan. 2011. Corticolimbic function in impulsive aggressive behavior. Biological psychiatry 69, 12 (2011), 1153-1159.
[33] Pamela M Cole. 1986. Children's spontaneous control of facial expression. Child development (1986), 1309-1321.
[34] Pamela M Cole, Carolyn Zahn-Waxler, Nathan A Fox, Barbara A Usher, and Jean D Welsh. 1996. Individual differences in emotion regulation and behavior problems in preschool children. Journal of Abnormal Psychology 105, 4 (1996), 518.
[35] Jean Costa, Alexander T Adams, Malte F Jung, Frangois Guimbretiere, and Tanzeem Choudhury. 2016. EmotionCheck: leveraging bodily signals and false feedback to regulate our emotions. In Proceedings of the 2016 ACM International Joint Conference on Pervasive and Ubiquitous Computing. 758-769.
[36] Ana Cubillo, Rozmin Halari, Anna Smith, Eric Taylor, and Katya Rubia. 2012. A review of fronto-striatal and fronto-cortical brain abnormalities in children and adults with Attention Deficit Hyperactivity Disorder (ADHD) and new evidence for dysfunction in adults with ADHD during motivation and attention. cortex 48, 2 (2012), 194-215.
[37] Richard J Davidson, Paul Ekman, Clifford D Saron, Joseph A Senulis, and Wallace V Friesen. 1990. Approach-withdrawal and cerebral asymmetry: emotional expression and brain physiology: I. Journal of personality and social psychology 58, 2 (1990), 330.
[38] Munmun De Choudhury, Michael Gamon, Scott Counts, and Eric Horvitz. 2013. Predicting depression via social media. Icwsm 13 (2013), 1-10.
[39] Munmun De Choudhury, Emre Kiciman, Mark Dredze, Glen Coppersmith, and Mrinal Kumar. 2016. Discovering shifts to suicidal ideation from mental health content in social media. In Proceedings of the 2016 CHI conference on human factors in computing systems. 2098-2110.
[40] Nuria de la Osa, Roser Granero, Esther Trepat, Josep Maria Domenech, and Lourdes Ezpeleta. 2016. The discriminative capacity of CBCL/11/2-5-DSM5 scales to identify disruptive and internalizing disorders in preschool children. European child & adolescent psychiatry 25, 1 (2016), 17-23.
[41] Minet de Wied, Anton van Boxtel, Ruud Zaalberg, Paul P Goudena, and Walter Matthys. 2006. Facial E M G responses to dynamic emotional facial expressions in boys with disruptive behavior disorders. Journal of Psychiatric research 40, 2 (2006), 112-121.
[42] Thomas G Dietterich, Richard H Lathrop, and Tomis Lozano-Perez. 1997. Solving the multiple instance problem with axis-parallel rectangles. Artificial intelligence 89, 1-2 (1997), 31-71.
[43] Hanna Drimalla, Niels Landwehr, Irina Baskow, Behnoush Behnia, Stefan Roepke, Isabel Dziobek, and Tobias Scheffer. 2018. Detecting autism by analyzing a simulated social interaction. In Joint European Conference on Machine Learning and Knowledge Discovery in Databases. Springer, 193-208.
[44] George J DuPaul, Robert Reid, Arthur D Anastopoulos, Matthew C Lambert, Marley W Watkins, and Thomas J Power. 2016. Parent and teacher ratings of attention-deficit/hyperactivity disorder symptoms: Factor structure and normative data. Psychological Assessment 28, 2 (2016), 214.
[45] Damien Dupré, Eva G Krumhuber, Dennis Küster, and Gary J McKeown. 2020. A performance comparison of eight commercially available automatic classifiers for facial affect recognition. Plos one 15, 4 (2020), e0231968.
[46] Nancy Eisenberg and Richard A Fabes. 1992. Emotion, regulation, and the development of social competence. (1992).
[47] Paul Ekman, Richard J Davidson, and Wallace V Friesen. 1990. The Duchenne smile: emotional expression and brain physiology: II. Journal of personality and social psychology 58, 2 (1990), 342.
[48] P Ekman and WV Friesen. 1978. Facial Action Coding System (FACS): Manual. Palo Alto.
[49] Paul Ekman and Wallace V Friesen. 1971. Constants across cultures in the face and emotion. Journal of personality and social psychology 17, 2 (1971), 124.
[50] Jon D Elhai, Jason C Levine, and Brian J Hall. 2019. The relationship between anxiety symptom severity and problematic smartphone use: A review of the literature and conceptual frameworks. Journal of Anxiety Disorders 62 (2019), 45-52.
[51] Charles Fage. 2015. An emotion regulation app for school inclusion of children with ASD: design principles and preliminary results for its evaluation. ACM SIGACCESS Accessibility and Computing 112 (2015), 8-15.
[52] Tobias Fischer, Hyung Jin Chang, and Yiannis Demiris. 2018. Rt-gene: Real-time eye gaze estimation in natural environments. In
Proceedings of the European Conference on Computer Vision (ECCV). 334-352.
[53] Thomas B Fitzpatrick. 1988. The validity and practicality of sun-reactive skin types I through VI. Archives of dermatology 124, 6 (1988), 869-871.
[54] E Friesen and Paul Ekman. 1978. Facial action coding system: a technique for the measurement of facial movement. Palo Alto 3 (1978).
[55] Thomas Gärtner, Peter A Flach, Adam Kowalczyk, and Alexander J Smola. 2002. Multi-instance kernels. In ICML, Vol. 2. 7.
[56] Miles Gilliom, Daniel S Shaw, Joy E Beck, Michael A Schonberg, and JoElla L Lukon. 2002. Anger regulation in disadvantaged preschool boys: Strategies, antecedents, and the development of self-control. Developmental psychology 38, 2 (2002), 222.
[57] Adam S Grabell, Theodore J Huppert, Frank A Fishburn, Yanwei Li, Christina O Hlutkowsky, Hannah M Jones, Lauren S Wakschlag, and Susan B Perlman. 2019. Neural correlates of early deliberate emotion regulation: Young children's responses to interpersonal scaffolding. Developmental cognitive neuroscience 40 (2019), 100708.
[58] Adam S Grabell, Theodore J Huppert, Frank A Fishburn, Yanwei Li, Hannah M Jones, Aimee E Wilett, Lisa M Bemis, and Susan B Perlman. 2018. Using facial muscular movements to understand young children's emotion regulation and concurrent neural activation. Developmental science 21, 5 (2018), e12628.
[59] Adam S Grabell, Yanwei Li, Jeff W Barker, Lauren S Wakschlag, Theodore J Huppert, and Susan B Perlman. 2018. Evidence of non-linear associations between frustration-related prefrontal cortex activation and the normal: abnormal spectrum of irritability in young children. Journal of abnormal child psychology 46, 1 (2018), 137-147.
[60] Paulo A Graziano, Rachael D Reavis, Susan P Keane, and Susan D Calkins. 2007. The role of emotion regulation in children's early academic success. Journal of school psychology 45, 1 (2007), 3-19.
[61] James J Gross. 2014. Emotion regulation: Conceptual and empirical foundations. (2014).
[62] Helen Harris and Clifford Nass. 2011. Emotion regulation for frustrating driving contexts. In Proceedings of the SIGCHI Conference on Human Factors in Computing Systems. 749-752.
[63] Caroline Heary, Eilis Hennessy, Lorraine Swords, and Patrick Corrigan. 2017. Stigma towards mental health problems during childhood and adolescence: Theory, research and intervention approaches. Journal of Child and Family Studies 26, 11 (2017), 2949-2959.
[64] Aaron S Heller, Regina C Lapate, Kaitlyn E Mayer, and Richard J Davidson. 2014. The face of negative affect: trial-by-trial corrugator responses to negative pictures are positively associated with amygdala and negatively associated with ventromedial prefrontal cortex activity. Journal of Cognitive Neuroscience 26, 9 (2014), 2102-2110.
[65] Stefan G Hofmann, Alice T Sawyer, Angela Fang, and Anu Asnaani. 2012. Emotion dysregulation model of mood and anxiety disorders. Depression and anxiety 29, 5 (2012), 409-416.
[66] Tahera Hossain, Md Shafiqul Islam, Md Atiqur Rahman Ahad, and Sozo Inoue. 2019. Human activity recognition using earable device. In Adjunct Proceedings of the 2019 ACM International Joint Conference on Pervasive and Ubiquitous Computing and Proceedings of the 2019 ACM International Symposium on Wearable Computers. 81-84.
[67] Yu Huang, Haoyi Xiong, Kevin Leach, Yuyan Zhang, Philip Chow, Karl Fua, Bethany A Teachman, and Laura E Barnes. 2016. Assessing social anxiety using gps trajectories and point-of-interest data. In Proceedings of the 2016 ACM International Joint Conference on Pervasive and Ubiquitous Computing. 898-903.
[68] Jeremy F Huckins, Alex W DaSilva, Rui Wang, Weichen Wang, Elin L Hedlund, Eilis I Murphy, Richard B Lopez, Courtney Rogers, Paul E Holtzheimer, William M Kelley, et al. 2019. Fusing mobile phone sensing and brain imaging to assess depression in college students. Frontiers in Neuroscience 13 (2019), 248.
[69] James J Hudziak, William Copeland, Catherine Stanger, and Martha Wadsworth. 2004. Screening for DSM-IV externalizing disorders with the Child Behavior Checklist: a receiver-operating characteristic analysis. Journal of child psychology and psychiatry 45, 7 (2004), 1299-1307.
[70] Daren C Jackson, Corrina J Mueller, Isa Dolski, Kim M Dalton, Jack B Nitschke, Heather L Urry, Melissa A Rosenkranz, Carol D Ryff, Burton H Singer, and Richard J Davidson. 2003. Now you feel it, now you don't: Frontal brain electrical asymmetry and individual differences in emotion regulation. Psychological science 14, 6 (2003), 612-617.
[71] Steven L Jacques. 2013. Optical properties of biological tissues: a review. Physics in Medicine & Biology 58, 11 (2013), R37.
[72] Sue Jamison-Powell, Conor Linehan, Laura Daley, Andrew Garbett, and Shaun Lawson. 2012. “I can't get no sleep” discussing #insomnia on twitter. In Proceedings of the SIGCHI Conference on Human Factors in Computing Systems. 1501-1510.
[73] Herbert H Jasper. 1958. The ten-twenty electrode system of the International Federation. Electroencephalogr. Clin. Neurophysiol. 10 (1958), 370-375.
[74] Amanda Jensen-Doss and Kristin M Hawley. 2010. Understanding barriers to evidence-based assessment: Clinician attitudes toward standardized assessment tools. Journal of Clinical Child & Adolescent Psychology 39, 6 (2010), 885-896.
[75] Bihan Jiang, Michel F Valstar, and Maja Pantic. 2011. Action unit detection using sparse appearance descriptors in space-time video volumes. In Face and Gesture 2011. IEEE, 314-321.
[76] Joohwan Kim, Michael Stengel, Alexander Majercik, Shalini De Mello, David Dunn, Samuli Laine, Morgan McGuire, and David Luebke. 2019. Nvgaze: An anatomically-informed dataset for low-latency, near-eye gaze estimation. In Proceedings of the 2019 CHI Conference on Human Factors in Computing Systems. 1-12.
[77] M Justin Kim, Rebecca A Loucks, Amy L Palmer, Annemarie C Brown, Kimberly M Solomon, Ashley N Marchante, and Paul J Whalen. 2011. The structural and functional connectivity of the amygdala: from normal emotion to pathological anxiety. Behavioural brain research 223, 2 (2011), 403-410.
[78] Michael Koenigs and Jordan Grafman. 2009. The functional neuroanatomy of depression: distinct roles for ventromedial and dorsolateral prefrontal cortex. Behavioural brain research 201, 2 (2009), 239-243.
[79] Yubo Kou and Xinning Gui. 2020. Emotion Regulation in eSports Gaming: A Qualitative Study of League of Legends. Proceedings of the ACM on Human-Computer Interaction 4, CSCW2 (2020), 1-25.
[80] Maria Kovacs, Joel Sherrill, Charles J George, Myrna Pollock, Rameshwari V Tumuluru, and Vincent Ho. 2006. Contextual emotion-regulation therapy for childhood depression: Description and pilot testing of a new intervention. Journal of the American Academy of Child & Adolescent Psychiatry 45, 8 (2006), 892-903.
[81] Ellen Leibenluft, Dennis S Charney, and Daniel S Pine. 2003. Researching the pathophysiology of pediatric bipolar disorder. Biological Psychiatry 53, 11 (2003), 1009-1020.
[82] Astar Lev, Yoram Braw, Tomer Elbaum, Michael Wagner, and Yuri Rassovsky. 2020. Eye Tracking During a Continuous Performance Test: Utility for Assessing ADHD Patients. Journal of Attention Disorders (2020), 1087054720972786.
[83] Valentina Levantini, Pietro Muratori, Emanuela Inguaggiato, Gabriele Masi, Annarita Milone, Elena Valente, Alessandro Tonacci, and Lucia Billeci. 2020. EYES are the window to the mind: Eye-tracking technology as a novel approach to study clinical characteristics of ADHD. Psychiatry Research 290 (2020), 113135.
[84] Deborah L Levy, Anne B Sereno, Diane C Gooding, and Gilllian A O'Driscoll. 2010. Eye tracking dysfunction in schizophrenia: characterization and pathophysiology. In Behavioral Neurobiology of Schizophrenia and Its Treatment. Springer, 311-347.
[85] Yan Li, David M J Tax, Robert P W Duin, and Marco Loog. 2013. Multiple-instance learning as a classifier combining problem. Pattern recognition 46, 3 (2013), 865-874.
[86] Hong Lu, Denise Frauendorfer, Mashfiqui Rabbi, Marianne Schmid Mast, Gokul T Chittaranjan, Andrew T Campbell, Daniel Gatica-Perez, and Tanzeem Choudhury. 2012. Stresssense: Detecting stress in unconstrained acoustic environments using smartphones. In Proceedings of the 2012 ACM conference on ubiquitous computing. 351-360.
[87] Diana MacLean, Asta Roseway, and Mary Czerwinski. 2013. MoodWings: a wearable biofeedback device for real-time stress intervention. In Proceedings of the 6th international conference on PErvasive Technologies Related to Assistive Environments. 1-8.
[88] Eric J Mash and John Hunsley. 2005. Evidence-based assessment of child and adolescent disorders: Issues and challenges. Journal of clinical child and adolescent psychology 34, 3 (2005), 362-379.
[89] Mark Matthews, Saeed Abdullah, Elizabeth Murnane, Stephen Voida, Tanzeem Choudhury, Geri Gay, and Ellen Frank. 2016. Development and evaluation of a smartphone-based measure of social rhythms for bipolar disorder. Assessment 23, 4 (2016), 472-483.
[90] Addison Mayberry, Pan Hu, Benjamin Marlin, Christopher Salthouse, and Deepak Ganesan. 2014. iShadow: design of a wearable, real-time mobile gaze tracker. In Proceedings of the 12th annual international conference on Mobile systems, applications, and services. 82-94.
[91] Katie A McLaughlin, Matthew Peverill, Andrea L Gold, Sonia Alves, and Margaret A Sheridan. 2015. Child maltreatment and neural systems underlying emotion regulation. Journal of the American Academy of Child & Adolescent Psychiatry 54, 9 (2015), 753-762.
[92] Douglas S Mennin, Richard G Heimberg, Cynthia L Turk, and David M Fresco. 2002. Applying an emotion regulation framework to integrative approaches to generalized anxiety disorder. Clinical Psychology: Science and Practice 9, 1 (2002), 85-90.
[93] Margaret Mitchell, Simone Wu, Andrew Zaldivar, Parker Barnes, Lucy Vasserman, Ben Hutchinson, Elena Spitzer, Inioluwa Deborah Raji, and Timnit Gebru. 2019. Model cards for model reporting. In Proceedings of the conference on fairness, accountability, and transparency. 220-229.
[94] Philipp Mock, Maike Tibus, Ann-Christine Ehlis, Harald Baayen, and Peter Gerjets. 2018. Predicting ADHD risk from touch interaction data. In Proceedings of the 20th ACM International Conference on Multimodal Interaction. 446-454.
[95] David C Mohr, Mi Zhang, and Stephen M Schueller. 2017. Personal sensing: understanding mental health using ubiquitous sensors and machine learning. Annual review of clinical psychology 13 (2017), 23-47.
[96] Amir Muaremi, Franz Gravenhorst, Agnes Grünerbl, Bert Arnrich, and Gerhard Tröster. 2014. Assessing bipolar episodes using speech cues derived from phone calls. In International Symposium on Pervasive Computing Paradigms for Mental Health. Springer, 103-114.
[97] Åsa Nilsonne. 1987. Acoustic analysis of speech variables during depression and after improvement. Acta Psychiatrica Scandinavica 76, 3 (1987), 235-245.
[98] Mikio Obuchi, Jeremy F Huckins, Weichen Wang, Alex daSilva, Courtney Rogers, Eilis Murphy, Elin Hedlund, Paul Holtzheimer, Shayan Mirjafari, and Andrew Campbell. 2020. Predicting Brain Functional Connectivity Using Mobile Sensing. Proceedings of the ACM on Interactive, Mobile, Wearable and Ubiquitous Technologies 4, 1 (2020), 1-22.
[99] Averil Overton, Susan Selway, Kenneth Strongman, and Michelle Houston. 2005. Eating disorders—The regulation of positive as well as negative emotion experience. Journal of Clinical Psychology in Medical Settings 12, 1 (2005), 39-56.
[100] Asli Ozdas, Richard G Shiavi, Stephen E Silverman, Marilyn K Silverman, and D Mitchell Wilkes. 2004. Investigation of vocal jitter and glottal flow spectrum as possible cues for depression and near-term suicidal risk. IEEE Transactions on Biomedical Engineering 51, 9 (2004), 1530-1540.
[101] Maja Pantic and Leon J. M. Rothkrantz. 2000. Automatic analysis of facial expressions: The state of the art. IEEE Transactions on pattern analysis and machine intelligence 22, 12 (2000), 1424-1445.
[102] Pablo Paredes and Matthew Chan. 2011. CalmMeNow: exploratory research and design of stress mitigating mobile interventions. In CHI'11 Extended Abstracts on Human Factors in Computing Systems. 1699-1704.
[103] Susan B Perlman, Beatriz Luna, Tyler C Hein, and Theodore J Huppert. 2014. fNIRS evidence of prefrontal regulation of frustration in early childhood. Neuroimage 85 (2014), 326-334.
[104] Skyler Place, Danielle Blanch-Hartigan, Channah Rubin, Cristina Gorrostieta, Caroline Mead, John Kane, Brian P Marx, Joshua Feast, Thilo Deckersbach, Andrew Nierenberg, et al. 2017. Behavioral indicators on a mobile sensing platform predict clinically validated psychiatric symptoms of mood and anxiety disorders. Journal of medical Internet research 19, 3 (2017), e75.
[105] Joseph S Raiker, Andrew J Freeman, Guillermo Perez-Algorta, Thomas W Frazier, Robert L Findling, and Eric A Youngstrom. 2017. Accuracy of Achenbach scales in the screening of attention-deficit/hyperactivity disorder in a community mental health clinic. Journal of the American Academy of Child & Adolescent Psychiatry 56, 5 (2017), 401-409.
[106] Michael P Reiman, Adam P Goode, Eric J Hegedus, Chad E Cook, and Alexis A Wright. 2013. Diagnostic accuracy of clinical tests of the hip: a systematic review with meta-analysis. British journal of sports medicine 47, 14 (2013), 893-902.
[107] Soha Rostaminia, Alexander Lamson, Subhransu Maji, Tauhidur Rahman, and Deepak Ganesan. 2019. W! NCE: Unobtrusive Sensing of Upper Facial Action Units with EOG-based Eyewear. Proceedings of the ACM on Interactive, Mobile, Wearable and Ubiquitous Technologies 3, 1 (2019), 1-26.
[108] Carolyn Saarni. 1979. Children's understanding of display rules for expressive behavior. Developmental psychology 15, 4 (1979), 424.
[109] Sohrab Saeb, Emily G Lattie, Stephen M Schueller, Konrad P Kording, and David C Mohr. 2016. The relationship between mobile phone location sensor data and depressive symptom severity. PeerJ 4 (2016), e2537.
[110] Akane Sano and Rosalind W Picard. 2013. Stress recognition using wearable sensors and mobile phones. In 2013 Humaine Association Conference on Affective Computing and Intelligent Interaction. IEEE, 671-676.
[111] Evangelos Sariyanidi, Hatice Gunes, and Andrea Cavallaro. 2014. Automatic analysis of facial affect: A survey of registration, representation, and recognition. IEEE transactions on pattern analysis and machine intelligence 37, 6 (2014), 1113-1133.
[112] Jocelyn Scheirer, Raul Fernandez, and Rosalind W Picard. 1999. Expression glasses: a wearable device for facial expression recognition. In CHI'99 Extended Abstracts on Human Factors in Computing Systems. 262-263.
[113] Jessica Schroeder, Chelsey Wilkes, Kael Rowan, Arturo Toledo, Ann Paradiso, Mary Czerwinski, Gloria Mark, and Marsha M Linehan. 2018. Pocket skills: A conversational mobile web app to support dialectical behavioral therapy. In Proceedings of the 2018 CHI Conference on Human Factors in Computing Systems. 1-15.
[114] Philip Shaw, Argyris Stringaris, Joel Nigg, and Ellen Leibenluft. 2014. Emotion dysregulation in attention deficit hyperactivity disorder. American Journal of Psychiatry 171, 3 (2014), 276-293.
[115] TO Smith, T Back, AP Toms, and CB Hing. 2011. Diagnostic accuracy of ultrasound for rotator cuff tears in adults: a systematic review and meta-analysis. Clinical radiology 66, 11 (2011), 1036-1048.
[116] David M J Tax, Marco Loog, Robert P W Duin, Veronika Cheplygina, and Wan-Jui Lee. 2011. Bag dissimilarities for multiple instance learning. In International Workshop on Similarity-Based Pattern Recognition. Springer, 222-234.
[117] Alina Trifan, Maryse Oliveira, and José Luis Oliveira. 2019. Passive sensing of health outcomes through smartphones: systematic review of current solutions and possible limitations. JMIR mHealth and uHealth 7, 8 (2019), e12649.
[118] Talia Tron, Abraham Peled, Alexander Grinsphoon, and Daphna Weinshall. 2015. Automated facial expressions analysis in schizophrenia: A continuous dynamic approach. In International Symposium on Pervasive Computing Paradigms for Mental Health. Springer, 72-81.
[119] Heather L Urry, Carien M Van Reekum, Tom Johnstone, Ned H Kalin, Marchell E Thurow, Hillary S Schaefer, Cory A Jackson, Corrina J Frye, Lawrence L Greischar, Andrew L Alexander, et al. 2006. Amygdala and ventromedial prefrontal cortex are inversely coupled during regulation of negative affect and predict the diurnal pattern of cortisol secretion among older adults. Journal of Neuroscience 26, 16 (2006), 4415-4425.
[120] Carien M van Reekum, Tom Johnstone, Heather L Urry, Marchell E Thurow, Hillary S Schaefer, Andrew L Alexander, and Richard J Davidson. 2007. Gaze fixations predict brain activation during the voluntary regulation of picture-induced negative affect. Neuroimage 36, 3 (2007), 1041-1055.
[121] Lauren S Wakschlag, Seung W Choi, Alice S Carter, Heide Hullsiek, James Burns, Kimberly McCarthy, Ellen Leibenluft, and Margaret J Briggs-Gowan. 2012. Defining the developmental parameters of temper loss in early childhood: implications for developmental psychopathology. Journal of Child Psychology and Psychiatry 53, 11 (2012), 1099-1108.
[122] Lauren S Wakschlag, Ryne Estabrook, Amelie Petitclerc, David Henry, James L Burns, Susan B Perlman, Joel L Voss, Daniel S Pine, Ellen Leibenluft, and Margaret L Briggs-Gowan. 2015. Clinical implications of a dimensional approach: the normal: abnormal spectrum of early irritability. Journal of the American Academy of Child & Adolescent Psychiatry 54, 8 (2015), 626-634.
[123] Lauren S Wakschlag, Patrick H Tolan, and Bennett L Leventhal. 2010. Research Review: ‘Ain't misbehavin’: Towards a developmentally-specified nosology for preschool disruptive behavior. Journal of Child Psychology and Psychiatry 51, 1 (2010), 3-22.
[124] Sebastian Walther, Katharina Stegmayer, Helge Horn, Nadja Razavi, Thomas J Müller, and Werner Strik. 2015. Physical activity in schizophrenia is higher in the first episode than in subsequent ones. Frontiers in psychiatry 5 (2015), 191.
[125] Jun Wang and Jean-Daniel Zucker. 2000. Solving multiple-instance problem: A lazy learning approach. (2000).
[126] Rui Wang, Min S H Aung, Saeed Abdullah, Rachel Brian, Andrew T Campbell, Tanzeem Choudhury, Marta Hauser, John Kane, Michael Merrill, Emily A Scherer, et al. 2016. CrossCheck: toward passive sensing and detection of mental health changes in people with schizophrenia. In Proceedings of the 2016 ACM International Joint Conference on Pervasive and Ubiquitous Computing. 886-897.
[127] Rui Wang, Weichen Wang, Alex DaSilva, Jeremy F Huckins, William M Kelley, Todd F Heatherton, and Andrew T Campbell. 2018. Tracking depression dynamics in college students using mobile phone and wearable sensing. Proceedings of the ACM on Interactive, Mobile, Wearable and Ubiquitous Technologies 2, 1 (2018), 1-26.
[128] Erin M Warnick, Michael B Bracken, and Stanislav Kasl. 2008. Screening efficiency of the Child Behavior Checklist and Strengths and Difficulties Questionnaire: A systematic review. Child and Adolescent Mental Health 13, 3 (2008), 140-147.
[129] Sarah Watts, Anna Mackenzie, Cherian Thomas, Al Griskaitis, Louise Mewton, Alishia Williams, and Gavin Andrews. 2013. CBT for depression: a pilot RCT comparing mobile phone vs. computer. BMC psychiatry 13, 1 (2013), 49.
[130] Nils Weidmann, Eibe Frank, and Bernhard Pfahringer. 2003. A two-level learning method for generalized multi-instance problems. In European Conference on Machine Learning. Springer, 468-479.
[131] Daniel G Whitney and Mark D Peterson. 2019. US national and state-level prevalence of mental health disorders and disparities of mental health care use in children. JAMA pediatrics 173, 4 (2019), 389-391.
[132] C Winograd-Gurvich, Nellie Georgiou-Karistianis, Paul Bernard Fitzgerald, Lynette Millist, and Owen B White. 2006. Ocular motor differences between melancholic and non-melancholic depression. Journal of affective disorders 93, 1-3 (2006), 193-203.
[133] Jia Wu, Shirui Pan, Xingquan Zhu, Chengqi Zhang, and Xindong Wu. 2018. Multi-instance learning with discriminative bag mapping. IEEE Transactions on Knowledge and Data Engineering 30, 6 (2018), 1065-1080.
[134] Zhefan Ye, Yin Li, Alireza Fathi, Yi Han, Agata Rozga, Gregory D Abowd, and James M Rehg. 2012. Detecting eye contact using wearable eye-tracking glasses. In Proceedings of the 2012 ACM conference on ubiquitous computing. 699-704.
[135] JungKyoon Yoon, Shuran Li, Yu Hao, and Chajoong Kim. 2019. Towards Emotional Well-Being by Design: 17 Opportunities for Emotion Regulation for User-Centered Healthcare Design. In Proceedings of the 13th EAI International Conference on Pervasive Computing Technologies for Healthcare. 351-355.
[136] Anis Zaman, Boyu Zhang, Vincent Silenzio, Ehsan Hoque, and Henry Kautz. 2020. Estimating Anxiety based on individual level engagements on YouTube & Google Search Engine. arXiv preprint arXiv:2007.00613 (2020).
[137] Janice Zeman and Judy Garber. 1996. Display rules for anger, sadness, and pain: It depends on who is watching. Child development 67, 3 (1996), 957-973.
[138] Cha Zhang, John Platt, and Paul Viola. 2005. Multiple instance boosting for object detection. Advances in neural information processing systems 18 (2005), 1417-1424.
[139] Qi Zhang and Sally A Goldman. 2001. EM-DD: An improved multiple-instance learning technique. In Advances in neural information processing systems. 1073-1080.
Zhi-Hua Zhou and Min-Ling Zhang. 2007. Solving multi-instance problems with classifier ensemble based on constructive clustering. Knowledge and information systems 11, 2 (2007), 155-170.

What is claimed is:

1. A method of predicting neural activation and psychopathology, comprising:
a) providing a frustration-inducing activity to a subject;
b) with the frustration-inducing activity, providing a choice to the subject;
c) capturing the subject's choice;
d) providing feedback to the subject about the subject's choice in step c); and
e) capturing the subject's reaction to the feedback provided in step d).

2. The method of claim 1, further comprising repeating steps b) through e) over a predetermined number of trials.

3. The method of claim 2, further comprising f) aggregating the subject's negative reactions over the number of trials.

4. The method of claim 3, further comprising g) comparing the subject's reaction to past reactions.

5. The method of claim 1, wherein step e) comprises extracting the subject's facial features from the subject's reaction.

6. The method of claim 5, wherein the subject's facial features are selected from the group consisting of eye gaze, head pose, and facial action units.

7. The method of claim 5, further comprising determining the facial expression of the subject.

8. The method of claim 7, wherein the facial expression is selected from the group consisting essentially of smile and sneer.

9. The method of claim 1, wherein the feedback provided in step d) comprises negative feedback.

10. The method of claim 1, further comprising step h) providing a final prediction of a diagnosis neural activation and psychopathology.

11. A method of predicting neural activation and psychopathology, comprising:
a) providing a frustration-inducing activity to a subject;
b) with the frustration-inducing activity, providing a choice to the subject;
c) capturing the subject's choice;
d) providing one of positive or negative feedback to the subject about the subject's choice in step c);
e) capturing the subject's reaction to the feedback provided in step d);
f) aggregating the subject's negative reactions over the number of trials in steps b) through e);
g) comparing the subject's reaction to past reactions from the number of trials; and
h) providing a final prediction of a diagnosis neural activation and psychopathology.

12. The method of claim 11, wherein step e) comprises extracting the subject's facial features from the subject's reaction.

13. The method of claim 12, wherein the subject's facial features are selected from the group consisting of eye gaze, head pose, and facial action units.

14. The method of claim 12, further comprising determining the facial expression of the subject.

15. The method of claim 14, wherein the facial expression is selected from the group consisting essentially of smile and sneer.

16. A system for predicting neural activation and psychopathology, comprising:
a computing device having a storage, a memory, a processor, a camera, an input device, a display,
software operative to cause the processor to cause displayed on the display
a) providing a frustration-inducing activity to a subject using the computing device;
b) with the frustration-inducing activity, providing a choice to the subject on the display;
c) capturing the subject's choice with the input device;
d) providing one of positive or negative feedback to the subject about the subject's choice in step c) by causing the processor to show the feedback on the display;
e) with the camera, capturing the subject's reaction to the feedback provided in step d) and storing the subject's reaction in the memory or the storage;
f) with the computing device, aggregating the subject's negative reactions over the number of trials in steps b) through e);
g) with the computing device, comparing the subject's reaction to past reactions from the number of trials; and
h) with the computing device, providing a final prediction of a diagnosis neural activation and psychopathology.

17. The system of claim 16, wherein step e) comprises extracting, with the computing device, the subject's facial features from the subject's reaction.

18. The system of claim 17, wherein the subject's facial features are selected from the group consisting of eye gaze, head pose, and facial action units.

19. The system of claim 17, further comprising determining the facial expression of the subject.

20. The system of claim 19, wherein the facial expression is selected from the group consisting essentially of smile and sneer.
